(19) United States
(12) Patent Application Publication
(10) Pub. No.: US 2011/0251104 A1
(43) Pub. Date: Oct. 13, 2011

(54) COMPOSITIONS AND METHODS FOR DETECTING DEACETYLASE ACTIVITY

(76) Inventors: Can Ozbal, Lexington, MA (US); William LaMarr, Andover, MA (US)

(21) Appl. No.: 13/082,265

(22) Filed: Apr. 7, 2011

Related U.S. Application Data
(60) Provisional application No. 61/322,798, filed on Apr. 9, 2010.

(51) Int. Cl. C40B 30/10; C12Q 1/34

(52) U.S. Cl. 506/12; 435/18

(57) ABSTRACT

A method for the quantitative determination of the activity of enzymes in the Sirtuin family through the quantification of the acetyl-ADPr product that is formed is provided. The method described allows for a wide range of substrates, including peptides, intact proteins, or protein complexes (e.g. nucleosomes) to be used as substrates without the need for additional analytical methods to be developed.

### CROSS-REFERENCING

This application claims the benefit of provisional application Ser. No. 61/322,798, filed Apr. 9, 2010, which application is incorporated by reference herein for all purposes.

### BACKGROUND

Histone deacetylases regulate the expression of genes that are involved in cell signaling, regulation of the cell cycle, and human diseases, including cancer. Histone deacetylases (HDACs) vary in their cellular localization and mechanism of action and traditionally fall into one of three classes. Class I HDACs are found in most cell types and are localized almost exclusively in the nucleus. Class II HDACs are less ubiquitous than Class I HDACs, and shuttle between the nucleus and cytoplasm of the cell in response to specific signals. Class III HDACs, also known as sirtuins, couple their deacetylation activity to the hydrolysis of nicotimamide adenine dinucleotide (NAD⁺). Like a number of other HDACs, sirtuins deacetylate the lysine residues of many non-histone proteins, including transcription factors, synthetic enzymes, and components of cellular structure, thereby influencing cellular processes extending well beyond transcriptional silencing.

Sirtuin enzymes differ from other HDACs in their cellular localization, target substrates, and clinical significance. For example, SIRT1 is primarily found in the nucleus, where it can deacetylate multiple lysine residues of histone 1, histone 4, and p53, affecting metabolism, cellular differentiation, and apoptosis. In comparison, SIRT2 is found primarily in the cytoplasm where it deacetylates alpha-tubulin and has been linked to cancer pathogenesis. Due to the interplay between sirtuin function and a wide variety of human diseases, the action of these enzymes has become a promising target for drug discovery.

Studying sirtuin reactions by mass spectrometry (MS) provides for the direct detection of native molecules, minimizes artifacts that can arise when using modified substrates and eliminates additional steps needed to quantify product formation. Current MS methods detect deacetylation activity by monitoring the acetylation of a peptide substrate. Such methods require the development of specific methods for each substrate/product pair under study. It would be advantageous to have a single system that could detect the deacetylation activity of virtually any sirtuin substrate. This would eliminate the need to customize detection methods for each sirtuin substrate/product pair.

### SUMMARY

As described below, certain embodiments of the present invention provides mass spectrometric methods that provide for the detection of sirtuin activity by detecting production of the O-acetyl-ADP-ribose co-product, and related compositions. Because multiple sirtuins form this co-product, and it is formed via activity on any protein substrate, such methods can be used to detect the deacetylase activity of virtually any sirtuin. Such methods are particularly useful in screening for modulators of sirtuin activity.

In particular embodiments, the invention provides mass spectrometric methods for analyzing sirtuin activity by monitoring the production of an O-acetyl-ADP-ribose co-product and related compositions. Some embodiments of the invention provide screening methods that are useful for the development of highly specific drugs to treat a disease or disorder characterized by the methods delineated herein. In addition, certain methods of the invention provide a facile means to identify therapies that are safe for use in subjects. In addition, some methods of the invention provide a route for analyzing virtually any number of agents for effects on a disease described herein with high-volume throughput, high sensitivity, and low complexity.

### BRIEF DESCRIPTION OF THE DRAWINGS

FIG. 1 is a schematic diagram showing the general chemical reaction upon the deacetylation of a protein or peptide by a sirtuin. The reaction has 2 substrates (acetylated protein or peptide and NAD+) and three products (deactylated protein or peptide, 2′-O-acetyl-ADP-ribose, and nicotinamide).

FIG. 2 shows the chemical structure of 2′-O-acetyl-ADP-ribose.

FIG. 3 shows the chemical structure of ADP-ribose

FIG. 4 shows the daughter ion scan of ADP-ribose, used as the internal standard in this experiment, using a Sciex API4000 triple quadrupole mass spectrometer. The various fragments and the parent ion have been identified based on their respective molecular weights.

FIG. 5 shows the dose-response curve from a dilution series of ADP-ribose from 0.63 micromolar to zero micromolar (buffer-only sample) using a RapidFire 200 high-throughput mass spectrometry system interfaced to a AB Sciex API 4000 mass spectrometer. The signal is linear over the concentration range and the limit of quantification (LOQ) is below 10 nanomolar. The error bars represent the standard deviation from 6 independent measurements.

FIG. 6 shows the time and enzyme concentration dependent formation of acetyl-ADPr normalized to an internal standard consisting of 0.5 μM ADP-ribose. Various dilutions of the enzyme solution were used to determine the effect of enzyme concentration on reaction kinetics.

FIG. 7 shows the results of an experiment to determine the Kₘ of Sirt1 for the acetylated peptide substrate used in the reaction.

FIG. 8 shows the results of an experiment to determine the Kₘ of Sirt1 for NAD⁺ used in the reaction.

FIG. 9 shows the determination of the IC₅₀ value for suramin sodium, a known inhibitor of Sirt1. The IC₅₀ value was determined to be 4.0±0.1 μM.

FIG. 10 shows the determination of the IC₅₀ value for nicotinamide to determine the product inhibition in the reaction. The IC₅₀ value was determined to be 335 μM.

FIG. 11A is a schematic diagram showing NAD⁺ dependent protein deacetylases.

FIG. 11B is a schematic diagram illustrating the SPE-MS/MS Analysis.

FIGS. 12A-12C show a comparison of peptide based and acetyl-ADPr product based analysis. FIG. 12A FIG. 12A: SIRT1—Enzyme Titration Timecourse for Peptide Based Analysis. FIG. 12B: SIRT1—Enzyme Titration Timecourse for 2′-O-acetyl-ADP-ribose Based Analysis. FIG. 12C: SIRT1—Enzyme Linearity

FIG. 13 provides a comparison of peptide based and acetyl-ADPr product based analysis of the Km of p53 peptide. The graphs show determinations for p53 Peptide and NAD Co-substrates.

FIGS. 14A-14D provide a comparison of peptide based and acetyl-ADPr product based analysis of Sirt1, Sirt2, and Sirt3 activity. FIG. 14A SIRT1—IC₅₀ Determination for Nicotinamide. FIG. 14B: SIRT2—Enzyme Linearity, Kₘ of p53 Peptide and NAD⁺ Co-substrates, and IC₅₀ of Nicotinamide. FIG. 14C: SIRT3—Enzyme Linearity, Kₘ of H4 Peptide and NAD⁺ Co-substrates, and IC₅₀ of Nicotinamide. FIG. 14D-Comparison of Peptide Based and 2′-O-acetyl-ADP-ribose Based Assay Parameters

FIGS. 15A-15C show that the acetyl-ADPr product assay can be used for activation studies. FIG. 15A: Labeled Sirtuin Assay. FIG. 15B: “Label-free” Sirtuin Assay. FIG. 15C: SIRT1—Substrate Dependant Activation by Resveratrol.

FIGS. 16A-16D show that the acetyl-ADPr product assay can be used for the analysis of whole protein substrates. FIG. 16A: SIRT3—Deacetylation of Whole Histone (Sigma cat #: H4524). FIG. 16B: SIRT1—Deacetylation of Cytochrome C (Sigma cat #: C4186). FIG. 16C: SIRT1—Deacetylation of Full Length Human p53 (BlueSky cat #: 100394). FIG. 16D: SIRT5-Deacetylation of Cytochrome C (Sigma cat #: C4186).

FIG. 17 shows that the acetyl-ADPr product assay is useful for epigenetic screening applications of mass spectrometry.

### DEFINITIONS

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

“Laser desorption mass spectrometer” refers to a mass spectrometer that uses laser energy as a means to desorb, volatilize, and ionize an analyte.

“Tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector—magnetic sector mass spectrometers, and combinations thereof.

“Mass analyzer” refers to a sub-assembly of a mass spectrometer that comprises means for measuring a parameter that can be translated into mass-to-charge ratios of gas phase ions. In a time-of-flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd—Yag lasers and other pulsed laser sources. “Fluence” refers to the energy delivered per unit area of interrogated image. A high fluence source, such as a laser, will deliver about 1 mJ/mm2 to 50 mJ/mm2. Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

By “substrate” is meant the material on which an enzyme acts.

“Solid support” refers to a solid material which can be derivatized with, or otherwise attached to, a capture reagent. Exemplary solid supports include probes, microtiter plates and chromatographic resins.

Analyte” refers to any component of a sample that is desired to be detected. The term can refer to a single component or a plurality of components in the sample.

“Monitoring” refers to recording changes in a continuously varying parameter.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in analyte levels, preferably a 25% change, more preferably a 40% change, and most preferably a 50% or greater change in analyte levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include conditions such as cancer that are characterized by undesirable increases or decreases in sirtuin activity.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DETAILED DESCRIPTION

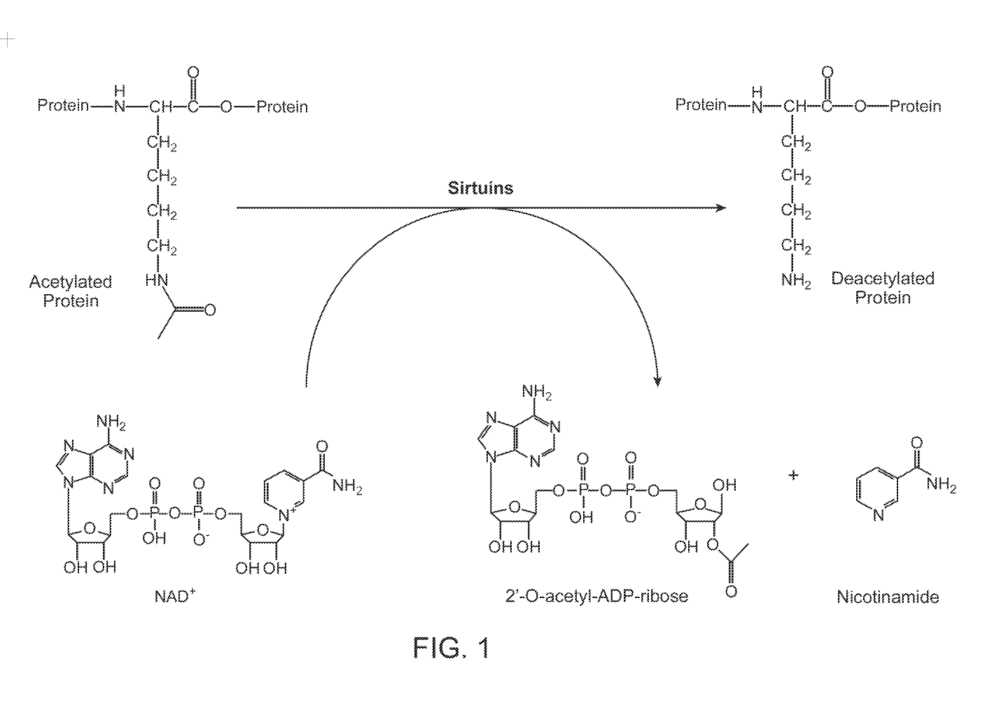

Some embodiments of the invention feature compositions and methods that are useful for assaying deacetylase activity by monitoring formation of 2′-O-acetyl-ADP-ribose (acetyl-ADPr). These embodiments are based, in part, on the discovery that acetyl-ADPr can be used to monitor the activity of virtually any sirtuin acting on virtually any substrate. Acetyl-ADPR is formed as a co-product with deacetylated protein or peptide at a ratio of 1:1. While the exact nature of the peptide or protein used in an in vitro experiment or as part of an in vivo cell or animal study may differ greatly, the acetyl-ADPr is a constant product. Furthermore, acetyl-ADPr is a novel metabolite enabling selective investigation of sirtuins in in vivo experiments. The ability to quantitatively detect the amount of acetyl-ADPr using mass spectrometry facilitates the interrogation of the level of activity of sirtuins in a wide range of applications. Due to the interplay between sirtuin function and a wide variety of human diseases, the action of sirtuin enzymes shown in FIG. 1 has become a promising target for drug discovery. In certain embodiments it may be necessary to use appropriate internal or external standards to accurately quantify the level of acetyl-ADPr in a sample. These standards could be added to the samples prior to analysis by mass spectrometry. In a one embodiment a known quantity of a stable isotope of acetyl-ADPr is added to sample prior to mass spectrometric analysis. The stable isotope could consist of acetyl-ADPr that includes one or more atoms of deuterium or carbon-13 in place of hydrogen or carbon-12. Alternately a stable isotope could also contain one or more atoms of oxygen-18 or nitrogen-15 instead of oxygen-16 or nitrogen-14. Because these stable isotopes are not naturally occurring in any great abundance, such an internal standard could be chemically synthesized and used in a variety of in vivo experiments.

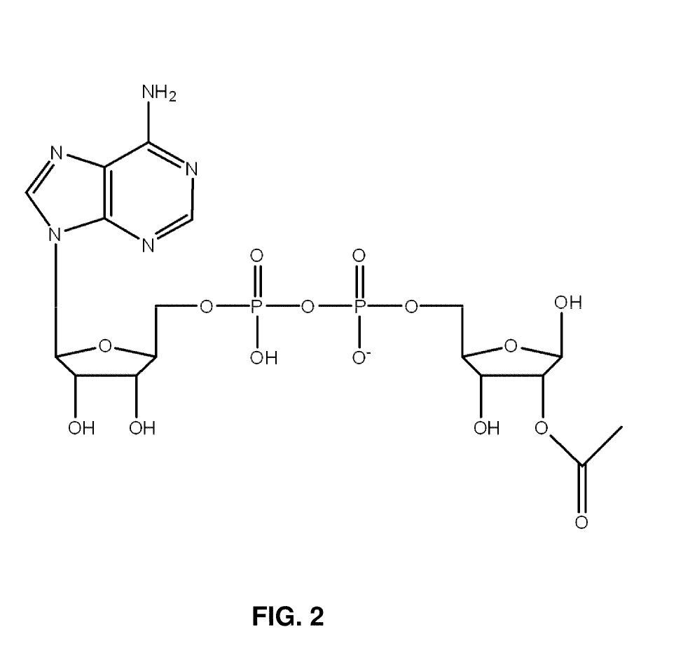

A much wider range of internal standards can be selected for use in simpler in vitro experiments where potential competition from mass spectrometric signal from endogenous material is less of a concern. In one embodiment, ADP-ribose (ADPr) can be used as an internal standard. The structural similarity of acetyl-ADPr and ADPr can be seen in FIG. 2. ADPr is commercially available from multiple suppliers precluding the need for custom chemical or enzymatic synthesis. It should be apparent to those skilled in the art that there is a very wide range of possible molecules that can be used as an internal standard for the purpose of accurate quantification of acetyl-ADPr by mass spectrometry in a sample.

There are many different types of mass spectrometers that could be used for the quantification of acetyl-ADPr and/or the internal standard(s) used in the analysis. Examples include single or triple quadrupole (QqQ) systems, trap-based systems including linear or orbital traps, time-of-flight (ToF) based systems, and hybrid systems such as Q-TOF's and TOF-TOF's. Furthermore, there are different types of ionization techniques that can be used for the ionization of the analytes of interest, including electrospray ionization (ESI), atmospheric pressure chemical ionization (APCI), atmospheric pressure photoionization (APPI), matrix-assisted laser desorption ionization (MALDI), surface-enhanced laser desorption (SELDI), desorption electrospray ionization (DESI), and others. It is understood that one skilled in the art could develop methods for the quantification of acetyl-ADPr and selected internal standards using various mass spectrometry platforms and ionization techniques.

In one embodiment the mass spectrometric analysis of the levels of acetyl-ADPr and internal standard in individual samples for would be performed in a high-throughput fashion to facilitate drug discovery and drug development programs in biopharmaceutical research. There are multiple mass spectrometry-based systems that are commercialized as high-throughput platforms. Commercially available systems include the Aria platform from ThermoFisher Scientific, FlashQuant from AB Sciex, LDTD from Phytronix, HPLC from Waters Corporation, UHPLC from Agilent Technologies, RapidFire Mass Spectrometry from Biocius Life Sciences, and others. The method of using mass spectrometry for quantifying acetyl-ADPr described in the current invention is applicable to any of the high-throughput mass spectrometric approaches described above.

The current invention allows for the quantitative determination of the activity of enzymes in the Sirtuin family through the quantification of the acetyl-ADPr product that is formed. The methods described herein allow for a wide range of substrates, including peptides, intact proteins, or protein complexes (e.g.: nucleosomes) to be used as substrates without the need for additional analytical methods to be developed.

### Mass Spectrometry Assays for Acetyltransferase/Deacetylase Activity

Provided herein are methods for determining the activity of NAD-dependent deacetylase enzymes (e.g., SIRT1, 2, 3, 5). The methods may involve, for example, contacting a substrate with an NAD-dependent Sirt and detecting acetyl-ADPr product using mass spectrometry. In other embodiments, the invention provides methods for identifying agents that modulate the activity of NAD-dependent deacetylase enzymes (e.g., SIRT1, 2, 3, 5). The methods may involve, for example, contacting a substrate with a SIRT enzyme in the presence of a test agent and detecting acetyl-ADPr product using mass spectrometry.

In certain embodiments, the activity of a NAD-dependent deacetylase enzyme (e.g., SIRT1, 2, 3, 5) is determined using the methods described herein. In particular embodiments, the activity of a deacetylase enzyme may be determined using the methods described herein. A deacetylase is an enzyme that releases an acetyl group from an acetylated peptide. Exemplary deacetylase enzymes include, for example, histone deacetylases (HDACs) class I or II and HDACs class III (or sirtuins). Class I HDACs (HDACs 1, 2, 3 and 8) bear similarity to the yeast RPD3 protein, are located in the nucleus and are found in complexes associated with transcriptional co-repressors. Class II HDACs (HDACs 4, 5, 6, 7 and 9) are similar to the yeast HDA1 protein, and have both nuclear and cytoplasmic subcellular localization. Both Class I and II HDACs are inhibited by hydroxamic acid-based HDAC inhibitors, such as SAHA. Class III HDACs form a structurally distant class of NAD dependent enzymes that are related to the yeast SIR2 proteins and are not inhibited by hydroxamic acid-based HDAC inhibitors.

Deacetylases useful in the methods described herein include sirtuin proteins. A sirtuin protein refers to a member of the sirtuin deacetylase protein family, or preferably to the sir2 family, which include yeast Sir2 (GenBank Accession No. P53685), C. elegans Sir-2.1 (GenBank Accession No. NP_{—}501912), and human SIRT1 (GenBank Accession No. NM_{—}012238 and NP_{—}036370 (or AF083106)) and SIRT2 (GenBank Accession No. NM_{—}012237, NM_{—}030593, NP_{—}036369, NP_{—}085096, and AF083107) proteins. Other family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (Brachmann et al. (1995) Genes Dev. 9:2888 and Frye et al. (1999) BBRC 260:273). Homologs, e.g., orthologs and paralogs, domains, fragments, variants and derivatives of the foregoing may also be used in accordance with the methods described herein.

In an exemplary embodiment, the methods described herein may be used to determine the activity of a SIRT1 protein. A SIRT1 protein refers to a member of the sir2 family of sirtuin deacetylases. In one embodiment, a SIRT1 protein includes yeast Sir2 (GenBank Accession No. P53685), C. elegans Sir-2.1 (GenBank Accession No. NP.sub.—501912), human SIRT1 (GenBank Accession No. NM_{—}012238 or NP_{—}036370 (or AF083106)), and human SIRT2 (GenBank Accession No. NM_{—}012237, NM_{—}030593, NP_{—}036369, NP_{—}085096, or AF083107) proteins, and equivalents and fragments thereof. In another embodiment, a SIRT1 protein includes a polypeptide comprising a sequence consisting of, or consisting essentially of, the amino acid sequence set forth in GenBank Accession Nos. NP_{—}036370, NP_{—}501912, NP.sub.—085096, NP_{—}036369, or P53685. SIRT1 proteins include polypeptides comprising all or a portion of the amino acid sequence set forth in GenBank Accession Nos. NP_{—}036370, NP.sub.—501912, NP_{—}085096, NP_{—}036369, or P53685; the amino acid sequence set forth in GenBank Accession Nos. NP_{—}036370, NP_{—}501912, NP_{—}085096, NP_{—}036369, or P53685 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. NP_{—}036370, NP_{—}501912, NP_{—}085096, NP_{—}036369, or P53685, and functional fragments thereof. SIRT1 proteins also include homologs (e.g., orthologs and paralogs), variants, or fragments, of GenBank Accession Nos. NP_{—}036370, NP_{—}501912, NP 085096, NP_{—}036369, or P53685.

In one embodiment, the methods described herein may be used to determine the activity of a SIRT3 protein. A SIRT3 protein refers to a member of the sirtuin deacetylase protein family and/or to a homolog of a SIRT1 protein. In one embodiment, a SIRT3 protein includes human SIRT3 (GenBank Accession No. AAH01042, NP_{—}036371, or NP_{—}001017524) and mouse SIRT3 (GenBank Accession No. NP_{—}071878) proteins, and equivalents and fragments thereof. In another embodiment, a SIRT3 protein includes a polypeptide comprising a sequence consisting of, or consisting essentially of, the amino acid sequence set forth in GenBank Accession Nos. AAH01042, NP_{—}036371, NP_{—}001017524, or NP_{—}071878. SIRT3 proteins include polypeptides comprising all or a portion of the amino acid sequence set forth in GenBank Accession AAH01042, NP_{—}036371, NP_{—}001017524, or NP_{—}071878; the amino acid sequence set forth in GenBank Accession Nos. AAH01042, NP_{—}036371, NP_{—}001017524, or NP_{—}071878 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. AAH01042, NP_{—}036371, NP_{—}001017524, or NP_{—}071878, and functional fragments thereof. SIRT3 proteins also include homologs (e.g., orthologs and paralogs), variants, or fragments, of GenBank Accession Nos. AAH01042, NP_{—}036371, NP 001017524, or NP._{—}071878.

In another embodiment, a biologically active portion of a sirtuin may be used in accordance with the methods described herein. A biologically active portion of a sirtuin refers to a portion of a sirtuin protein having a biological activity, such as the ability to deacetylate. Biologically active portions of sirtuins may comprise the core domain of a sirtuin. Biologically active portions of SIRT1 having GenBank Accession No. NP_{—}036370 that encompass the NAD binding domain and the substrate binding domain, for example, may include without limitation, amino acids 62-293 of GenBank Accession No. NP_{—}036370, which are encoded by nucleotides 237 to 932 of GenBank Accession No. NM._{—}012238. Therefore, this region is sometimes referred to as the core domain. Other biologically active portions of SIRT1, also sometimes referred to as core domains, include about amino acids 261 to 447 of GenBank Accession No. NP_{—}036370, which are encoded by nucleotides 834 to 1394 of GenBank Accession No. NM 012238; about amino acids 242 to 493 of GenBank Accession No. NP_{—}036370, which are encoded by nucleotides 777 to 1532 of GenBank Accession No. NM._{—}012238; or about amino acids 254 to 495 of GenBank Accession No. NP_{—}036370, which are encoded by nucleotides 813 to 1538 of GenBank Accession No. NM_{—}012238. In another embodiment, a biologically active portion of a sirtuin may be a fragment of a SIRT3 protein that is produced by cleavage with a mitochondrial matrix processing peptidase (MPP) and/or a mitochondrial intermediate peptidase (MIP).

NAD-dependent deacetylase enzymes (e.g., SIRT1, 2, 3, 5) that may be used in accordance with the methods described herein may be endogenous proteins, recombinant proteins, purified proteins, or proteins present in a mixture, such as a cell or tissue lysate. In certain embodiments, suitable enzymes for use in accordance with the methods described herein may be purchased commercially or purified using standard procedures. For example, human SIRT1 (Catalog #SE-239), human SIRT2 (Catalog #SE-251) and human SIRT3 (Catalog #SE-270) may be purchased from Biomol International (Plymouth Meeting, Pa.). Methods for expression and purification of human SIRT1 and human SIRT3 are described, for example, in PCT Publication No. WO 2006/094239. In other embodiments, suitable enzymes for use in accordance with the methods described herein may be provided as part of a mixture, such as, for example, a cell or tissue lysate or fractionated lysate. Suitable lysates include raw lysates including all components of the cell or tissue or lysates from which one or more components have been removed, such as, for example, nucleic acids, insoluble materials, membrane materials, etc. The lysate may be obtained from a variety of sources such as a blood cell sample, tissue sample, or cell culture.

A wide variety of substrates (e.g., peptide, proteins) may be used in accordance with the methods described herein. Exemplary substrates for deacetylases include, for example, histones (e.g., H1, H2, H2A, H2B, H3 and H4), nonhistone chromatin proteins (e.g., HMG1, HMG2, Yeast Sin1, HMG14, HMG17, and HMG I(Y)), transcriptional activators (e.g., p53, c-Myb, GATA-1, EKLF, MyoD, E2F, dTCF, and HIV Tat), nuclear receptor coactivators (e.g., ACTR, SRC-1, TIF2), general transcription factors (e.g., TFIIE and TFIIF), importin-α7, Rch1, and α-tubulin. Substrates used in accordance with the methods described herein may comprise an entire substrate protein or a portion thereof containing at least one lysine residue. In certain embodiments, it may be desirable to modify the sequence of a substrate protein, or a fragment thereof, to add, remove and/or change the location of one or more lysine residues. For example, it may be desirable to have a substrate peptide that contains one or more lysine residues located only in desired locations within the substrate peptide, e.g., toward the center of the substrate, toward an end of the substrate (e.g., N-terminal or C-terminal end), having multiple lysine residues clustered together, having lysine residues spread across the peptide, etc. In certain embodiments, it may be desirable to have a substrate peptide that contains only a single lysine residue. One or more lysine residues may be removed from a peptide substrate sequence by replacing the amino acid residue with a different amino acid residue or by deleting the amino acid residue from the sequence without substitution of a different amino acid. In certain embodiments, one or more lysine residues may be replaced using a conservative amino acid substitution.

In exemplary embodiments, the invention provides a method for identifying compounds that activate a sirtuin protein, such as, for example, a SIRT1 protein. In such embodiments, the methods utilize a substrate peptide that is a sirtuin activatable substrate peptide. A sirtuin activatable substrate peptide may be identified using a variety of sirtuin assays, including for example, the mass spectrometry assay described herein. In certain embodiments, the sequence of a sirtuin activatable substrate peptide is derived from a known sirtuin substrate, such as, for example, an HMG protein, p53, c-Myb, GATA-1, EKLF, MyoD, E2F, dTCF, or HIV Tat, or a fragment thereof. In certain embodiments, a sirtuin activatable substrate peptide may be from about 5-100, about 10-100, about 10-75, about 10-50, about 20-100, about 20-75, about 20-50, about 20-30, or about 20-25 amino acids in length. In certain embodiments, a sirtuin activatable substrate peptide comprises at least one hydrophobic region. In certain embodiments, a hydrophobic region may be located at or near one or both ends of the sirtuin activatable substrate peptide, e.g., the N-terminal and/or C-terminal ends. A hydrophobic region may be naturally occurring in the sequence of the sirtuin activatable substrate peptide, e.g., at least a portion of a sirtuin substrate protein comprising a hydrophobic region may be used as the substrate peptide. In the alternative, or in addition, a hydrophobic region may be added to a sirtuin activatable substrate peptide. For example, a hydrophobic region may be added to a substrate peptide by modifying the sequence of the peptide to increase the number of hydrophobic amino acid residues in a desired region, e.g., by adding 1, 2, 3, 4, 5, 6, 7, 8, 9, 10, or more, hydrophobic amino acid residues to a peptide either by the addition of new amino acid residues or by the replacement of existing non-hydrophobic (or less hydrophobic) amino acid residues with hydrophobic (or more strongly hydrophobic) amino acid residues. In certain embodiments, a hydrophobic region may be region of about 2, 3, 4, 5, 6, 7, 8, 9, 10 or more hydrophobic amino acid residues in a contiguous or substantially contiguous stretch within the peptide. Hydrophobic amino acid residues include alanine, phenylalanine, glycine, isoleucine, leucine, methionine, proline, valine, and tryptophan. In exemplary embodiments, hydrophobic regions comprise one or more tryptophan, alanine and/or phenylalanine amino acid residues. Alternatively, a hydrophobic region may be added to a substrate peptide by chemically modifying the peptide to increase its hydrophobicity. For example, a hydrophobic region may be introduced into a peptide by covalently attaching a hydrophobic chemical moiety to the peptide. Examples of chemical moieties include, for example, fluorophores, such as, AF 350, AF 430, AF 488, AF 532, AF 546, AF 568, AF 594, AF 633, AF 647, AF 660, AF 680, dintrophenyl, AMCA, Cascade Blue, Marina Blue, Fluorescein/FITC, Oregon Green 488, Rhodamine Green, BODIPY FL, BODIPY TMR, BODIPY TR, Oregon Green 514, Rhodamine Red, Tetramethylrhodamine, Texas Red, BODIIPY 630/650, BODTPY 650/665, QSY 7, Fluor X, Cy2 bis, Cy3 mono, Cy3.5 mono, Cy5 mono, Cy5.5 mono, Cy7 mono, DEAC, R6G, TAMRA, and MR121. Methods for covalently modifying a peptide with a chemical moiety such as a fluorophore are known in the art, and thus, can be conducted according to conventional methods. In exemplary embodiments, the hydrophobic chemical moiety may be covalently linked or conjugated to the peptide so as not to interfere with acetylation or deacetylation of the lysine residue(s).

Substrate peptides that may be used in accordance with the methods described herein can be synthesized according to conventional methods. The substrate peptides may include naturally occurring peptides, peptides prepared by genetic recombination techniques, and synthetic peptides. The peptides may be fused with other peptides (for example, glutathione-S-transferase, HA tag, FLAG tag, etc.) for convenience of purification, etc. Further, the peptide may comprise structural units other than amino acids so long as it serves as a substrate for a deacetylase or acetyltransferase. Typically, the synthesis of a peptide is achieved by adding amino acids, residue by residue, from the carboxyl terminus of the amino acid sequence of interest. Further, some of the peptide fragments synthesized in that way may be linked together to from a larger peptide molecule. For measuring deacetylase activity, the substrate peptide needs to be acetylated before the reaction is conducted. An exemplary method of amino acid acetylation includes acetylation of amino acids, whose .alpha.-amino groups and side-chain amino groups are blocked with protecting groups, with acetic anhydride, N-hydroxysuccinimide acetate, or similar reagents. These acetylated amino acids are then used to synthesize peptides comprising acetylated lysine residues, for example, using the solid-phase method. Generally, acetylated peptides can be synthesized using a peptide synthesizer according to the Fmoc method. For example, commercial suppliers, who provide custom peptide synthesis services, can synthesize peptides having specified amino acid sequences comprising residues acetylated at predetermined positions.

In certain embodiments, a method for identifying an agent that modulates the activity of an NAD-dependent deacetylase enzyme (e.g., SIRT1, 2, 3, 5) is provided. The method may involve comparing the activity of a deacetylase in the presence of a test agent to the activity of the deacetylase in a control reaction. The control reaction may simply be a duplicate reaction in which the test compound is not included. Alternatively, the control reaction may be a duplicate reaction in the presence of a compound having a known effect on the deacetylase activity (e.g., an activator, an inhibitor, or a compound having no effect on enzyme activity).

Due to the flexibility available in designing peptide substrates for the mass spectrometry based methods described herein, it is possible to optimize the peptide substrates to provide a low apparent Km thus permitting a lower concentration of substrate to be used in association with the methods.

The methods described herein utilize mass spectrometry for determining the level of acetyl-ADPr product in a reaction. Mass spectrometry (or simply MS) encompasses any spectrometric technique or process in which molecules are ionized and separated and/or analyzed based on their respective molecular weights. Thus, mass spectrometry and MS encompass any type of ionization method, including without limitation electrospray ionization (ESI), atmospheric-pressure chemical ionization (APCI) and other forms of atmospheric pressure ionization (API), and laser irradiation. Mass spectrometers may be combined with separation methods such as gas chromatography (GC) and liquid chromatography (LC). GC or LC separates the components in a mixture, and the components are then individually introduced into the mass spectrometer; such techniques are generally called GC/MS and LC/MS, respectively. MS/MS is an analogous technique where the first-stage separation device is another mass spectrometer. In LC/MS/MS, the separation methods comprise liquid chromatography and MS. Any combination (e.g., GC/MS/MS, GC/LC/MS, GC/LC/MS/MS, etc.) of methods can be used to practice the methods described herein. In such combinations, MS can refer to any form of mass spectrometry; by way of non-limiting example, LC/MS encompasses LC/ESI MS and LC/MALDI-TOF MS. Thus, mass spectrometry and MS include without limitation APCI MS; ESI MS; GC MS; MALDI-TOF MS; LC/MS combinations; LC/MS/MS combinations; MS/MS combinations; etc. Other examples of MS include, for example, MALDI-TOF-TOF MS, MALDI Quadrupole-time-of-flight (Q-TOF) MS, electrospray ionization (ESI)-TOF MS, ESI-Q-TOF, ESI-TOF-TOF, ESI-ion trap MS, ESI Triple quadrupole MS, ESI Fourier Transform Mass Spectrometry (FTMS), MALDI-FTMS, MALDI-Ion Trap-TOF, ESI-Ion Trap TOF, surface-enhanced laser desorption/ionization (SELDI), MS/MS/MS, ESI-MS/MS, quadrupole time-of-flight mass spectrometer QqTOF MS, MALDI-QqTOFMS, ESI-QqTOF MS, and chip capillary electrophoresis (chip-CE)-QqTOF MS, etc.

It is sometimes necessary to prepare samples comprising an analyte of interest for MS. Such preparations include without limitation purification and/or buffer exchange. Any appropriate method, or combination of methods, can be used to prepare samples for MS. One type of MS preparative method is liquid chromatography (LC), including without limitation HPLC and RP-HPLC.

High-pressure liquid chromatography (HPLC) is a separative and quantitative analytical tool that is generally robust, reliable and flexible. Reverse-phase (RP) is a commonly used stationary phase that is characterized by alkyl chains of specific length immobilized to a silica bead support. RP-HPLC is suitable for the separation and analysis of various types of compounds including without limitation biomolecules, (e.g., glycoconjugates, proteins, peptides, and nucleic acids, and, with mobile phase supplements, oligonucleotides). One of the most important reasons that RP-HPLC has been the technique of choice amongst all HPLC techniques is its compatibility with electrospray ionization (ESI). During ESI, liquid samples can be introduced into a mass spectrometer by a process that creates multiple charged ions (Wilm et al., Anal. Chem. 68:1, 1996). However, multiple ions can result in complex spectra and reduced sensitivity.

In HPLC, peptides and proteins are injected into a column, typically silica based C18. An aqueous buffer is used to elute the salts, while the peptides and proteins are eluted with a mixture of aqueous solvent (water) and organic solvent (acetonitrile, methanol, propanol). The aqueous phase is generally HTLC grade water with 0.1% acid and the organic solvent phase is generally an HPLC grade acetonitrile or methanol with 0.1% acid. The acid is used to improve the chromatographic peak shape and to provide a source of protons in reverse phase LC/MS. The acids most commonly used are formic acid, trifluoroacetic acid, and acetic acid. In RP HPLC, compounds are separated based on their hydrophobic character. With an LC system coupled to the mass spectrometer through an ESI source and the ability to perform data-dependant scanning, it is now possible in at least some instances to distinguish proteins in complex mixtures containing more than 50 components without first purifying each protein to homogeneity. Where the complexity of the mixture is extreme, it is possible to couple ion exchange chromatography and RP-HPLC in tandem to identify proteins from mixtures containing in excess of 1,000 proteins.

A particular type of MS technique, matrix-assisted laser desorption time-of-flight mass spectrometry (MALDI-TOF MS) (Karas et al., Int. J. Mass Spectrom. Ion Processes 78:53, 1987), has received prominence in analysis of biological polymers for its desirable characteristics, such as relative ease of sample preparation, predominance of singly charged ions in mass spectra, sensitivity and high speed. MALDI-TOF MS is a technique in which a UV-light absorbing matrix and a molecule of interest (analyte) are mixed and co-precipitated, thus forming analyte:matrix crystals. The crystals are irradiated by a nanosecond laser pulse. Most of the laser energy is absorbed by the matrix, which prevents unwanted fragmentation of the biomolecule. Nevertheless, matrix molecules transfer their energy to analyte molecules, causing them to vaporize and ionize. The ionized molecules are accelerated in an electric field and enter the flight tube. During their flight in this tube, different molecules are separated according to their mass to charge (m/z) ratio and reach the detector at different times. Each molecule yields a distinct signal. The method, may be used for detection and characterization of biomolecules, such as proteins, peptides, oligosaccharides and oligonucleotides, with molecular masses between about 400 and about 500,000 Da, or higher. MALDI-MS is a sensitive technique that allows the detection of low (10.sup.-15 to 10.sup.-18 mole) quantities of analyte in a sample.

Electrospray ionization may be used for both very large and small molecules. The electrospray process produces multiply charged analytes, making it somewhat easier to detect larger analytes such as proteins. Also, small molecules can be measured readily in the absence of matrix. The MALDI process requires a matrix, which may make it more difficult to analyze small molecules, for example, with molecular weights of less than about 700 daltons.

With certain mass spectrometers, for example, MALDI-TOF, sensitivity decreases as the molecular weight of a molecule increases. For example, the detection sensitivity of molecules with molecular weights in the range of about 10,000 daltons may be an order of magnitude or more lower than detection sensitivity of molecules with molecular weights in the range of about 1,000 daltons. Use and detection of a coding moiety and/or labels with a different, for example lower, molecular weight than the analyte can therefore enhance the sensitivity of the assay. Sensitivity can also be increased by using a coding moiety and/or that is very amenable to ionization.

In electrospray mass spectrometry, sample introduction into a mass spectrometer such as a quadropole, an ion trap, a TOF, a FTICR, or a tandem mass spectrometer, the higher molecular weight compounds, for example, proteins are observed as ions having a variable number of charge states. While the multiple charge phenomenon increases sensitivity, the spectra are more complex and difficult to interpret. Use and detection of a coding moiety with a less complex mass spectrum than the analyte can therefore enhance the resolution of the assay.

Various mass spectrometers may be used in accordance with the methods described herein. Representative examples include: triple quadrupole mass spectrometers, magnetic sector instruments (magnetic tandem mass spectrometer, JEOL, Peabody, Mass.), ionspray mass spectrometers (Bruins et al., Anal Chem. 59:2642-2647, 1987), electrospray mass spectrometers (including tandem, nano- and nano-electrospray tandem) (Fenn et al., Science 246:64-71, 1989), laser desorption time-of-flight mass spectrometers (Karas and Hillenkamp, Anal. Chem. 60:2299-2301, 1988), and a Fourier Transform Ion Cyclotron Resonance Mass Spectrometer (Extrel Corp., Pittsburgh, Mass.).

For additional information regarding mass spectrometers, see, e.g., Principles of Instrumental Analysis, 3rd ed., Skoog, Saunders College Publishing, Philadelphia, 1985; Kirk-Othmer Encyclopedia of Chemical Technology, 4th ed. Vol. 15 (John Wiley & Sons, New York 1995), pp. 1071-1094; Chemushevich and Thomson (EP1006559); Verentchikov et al. (WO/0077823); Clemmer and Reilly (WO/0070335); Hager (WO/0073750); WO99/01889; G. Siuzdak, Mass Spectrometry for Biotechnology, Academic Press, N.Y., (1996); Krutchinsky et al., WO 99/38185; Shevchenko et al., (2000) Anal. Chem. 72: 2132-2141; Figeys et al., (1998) Rapid Comm'ns. Mass Spec. 12-1435-144; Li et al. (2000) Anal. Chem. 72: 599-609; Li et al. (2000) Tibtech 18:151-160; Rowley et al. (2000) Methods 20:383-397; Kuster and Mann (1998) Curr. Opin. Structural Biol. 8: 393-400.; Chait et al. (1993) Science 262:89-92; Keough et al. (1999) Proc. Natl. Acad. Sci. USA 96:7131-6; and Bergman (2000) EXS 88:133-44.

In an exemplary embodiment, the mass spectrometry based assay methods described herein are conducted in a high throughput manner as described in C. C. Ozbal, et al., Assay and Drug Development Technologies 2: 373-381 (2004). In certain embodiments, the high throughput mass spectrometry based assay methods described herein utilize an integrated microfluidic system which uses an atmospheric pressure ionization triple quadrupole mass spectrometer as the detection system with electrospray ionization (ESI) or atmospheric pressure chemical ionization (APCI).

### Screening

In certain embodiments, methods for screening for compounds that modulate activity of NAD-dependent deacetylase enzymes (e.g., SIRT1, 2, 3, 5) are provided. In certain embodiments, the methods described herein may be used to identify an agent that decreases or increases deacetylase activity by at least about 10%, 25%, 50%, 75%, 80%, 90%, or 100%, or more, relative to the absence of the test compound. In an exemplary embodiment, the methods described herein may be used to identify a sirtuin activating compound that increases deacetylase activity by at least about 10%, 25%, 50%, 75%, 80%, 90%, or 100%, or more, relative to the sirtuin activating activity of resveratrol.

Test agents can be pharmacologic agents already known in the art or can be agents previously unknown to have any pharmacological activity. The agents can be naturally occurring or designed in the laboratory. They can be isolated from microorganisms, animals, or plants, and can be produced recombinantly, or synthesized by chemical methods known in the art. If desired, test agents can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J Med. Chem. 37, 2678, 1994; Cho et al., Science 261, 1303, 1993; Carell et al., Angew. Chem. Int. Ed Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

Test compounds can be screened for the ability to modulate deacetylase activity using high throughput screening. Using high throughput screening, many discrete agents can be tested in parallel so that large numbers of test compounds can be quickly screened. The most widely established techniques utilize 96-well microtiter plates. In addition to the plates, many instruments, materials, pipettors, robotics, plate washers, and plate readers are commercially available to fit the 96-well format.

Alternatively, free format assays, or assays that have no physical barrier between samples, can be used. Assays involving free formats are described, for example, in Jayawickreme et al., Proc. Natl. Acad. Sci. U.S.A. 19, 1614-18 (1994); Chelsky, “Strategies for Screening Combinatorial Libraries: Novel and Traditional Approaches,” reported at the First Annual Conference of The Society for Biomolecular Screening in Philadelphia, Pa. (Nov. 7-10, 1995); and Salmon et al., Molecular Diversity 2, 57-63 (1996). Another high throughput screening method is described in Beutel et al., U.S. Pat. No. 5,976,813.

In another embodiment, a kit for measuring the activity of NAD-dependent deacetylase enzymes (e.g., SIRT1, 2, 3, 5) and screening for compounds that inhibit or enhance the activity of such enzymes as described herein is provided. Such a kit may be useful for research purposes, drug discovery, diagnostic purposes, etc.

In certain embodiments, a kit may comprise a substrate (as described above) and one or more of the following: a NAD-dependent deacetylase enzymes (e.g., SIRT1, 2, 3, 5), one or more test compounds, a positive control, a negative control, instructions for use, a reaction vessel, buffers, and other reagents useful for mass spectrometry analysis. In certain embodiments, each component, e.g., the substrate peptide, the deacetylase, and/or test compound, may be packaged separately. A kit may also contain reagents for performing mass spectrometry, e.g., solvent or matrix, and, optionally, instructions for use of the components of the kit in a mass spectrometry assay described above.

Respective components of the kit may be combined so as to realize a final concentration that is suitable for the reaction. Further, in addition to these components, the kit may comprise a buffer that gives a condition suitable for the reaction. The enzyme preparation and the substrate peptide may be combined with other components that stabilize proteins. For example, the kit components may be stored and/or shipped in the presence of about 1% BSA and about 1% polyols (e.g., sucrose or fructose) to prevent protein denaturation after lyophilization.

Each component of the kit can be provided in liquid form or dried form. Detergents, preservatives, buffers, and so on, commonly used in the art may be added to the components so long as they do not inhibit the measurement of the deacetylase or acetyltransferase activity.

Compounds that activate or inhibit the NAD-dependent deacetylase enzymes (e.g., SIRT1, 2, 3, 5), which can be selected according to the method for screening of the present invention, are useful as candidate compounds for antimicrobial substances, anti-cancer agents, and a variety of other uses. For example, compounds that activate a sirtuin deacetylase protein may be useful for increasing the lifespan of a cell, and treating and/or preventing a wide variety of diseases and disorders including, for example, diseases or disorders related to aging or stress, diabetes, obesity, neurodegenerative diseases, chemotherapeutic induced neuropathy, neuropathy associated with an ischemic event, ocular diseases and/or disorders, cardiovascular disease, blood clotting disorders, and inflammation. In other embodiments, sirtuin deacetylase inhibitors may be useful for a variety of therapeutic applications including, for example, increasing cellular sensitivity to stress, increasing apoptosis, treatment of cancer, stimulation of appetite, and/or stimulation of weight gain.

The practice of the present method may employ, unless otherwise indicated, conventional techniques of molecular biology (including recombinant techniques), microbiology, cell biology, biochemistry and immunology, which are well within the purview of the skilled artisan. Such techniques may be explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Characterization of ADP-Ribose Using Mass Spectrometry

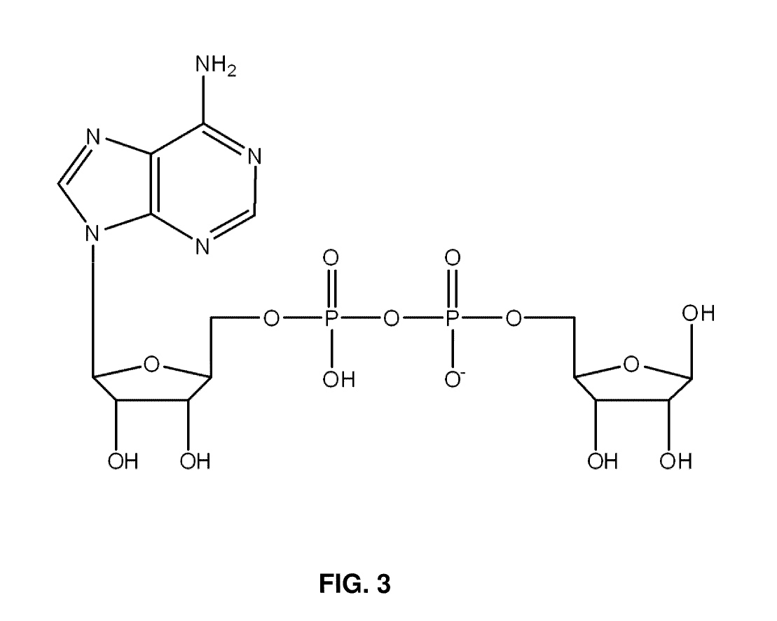

A high-throughput in vitro assay using the current invention was developed against a commercially available SIRT1 enzyme preparation. The assay was then used to fully characterize the enzyme preparation through a series of kinetic and end-point assays. Commercially available ADP-ribose was used to develop mass spectrometric methods and separation techniques for sensitive and selective detection in the assay. The structure of ADP-ribose is shown in FIG. 3. A quantitative analysis method for acetyl-ADPr, a compound that has a chemical structure and properties similar to ADP-ribose, was developed based on the method developed for ADP-ribose.

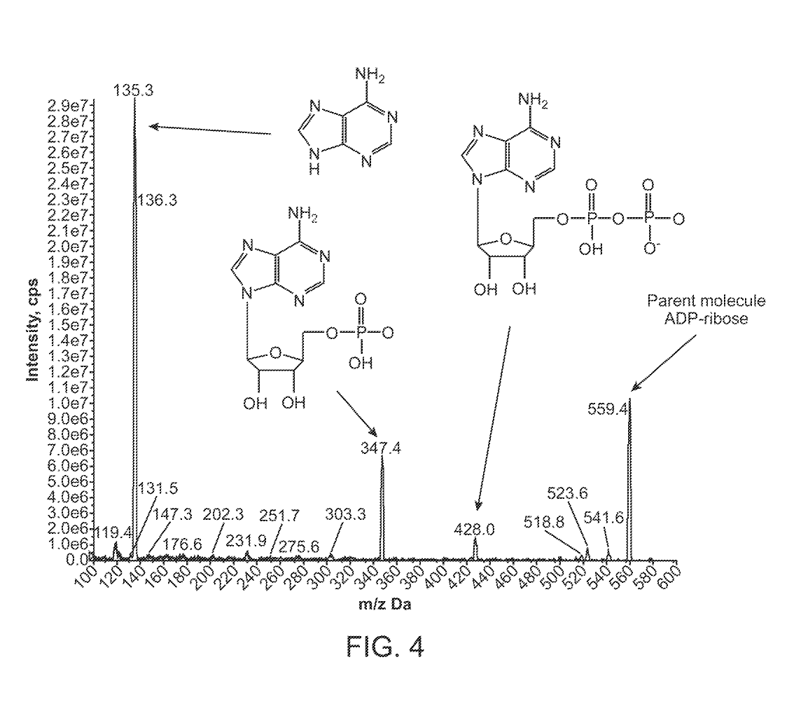

Initially a 1.0 micromolar solution of ADP-ribose in a 1:1 mixture or water:methanol acidified with the addition of 1% formic acid was infused into a Sciex API 4000 triple quarupole mass spectrometer at a flow rate of 50 μL/min using a syringe pump. The mass spectrometer was run in positive ion mode using electrospray ionization (ESI). This infusion was used to determine the mass spectrometric characteristics of ADP-ribose including the fragmentation pattern of the molecule, shown in FIG. 4. A molecular mass of 560.2 amu was determined for the parent molecule and a fragment at 135.2 amu (corresponding to the nicotinamide base) at a fragmentation energy of 50 Volts was selected for quantification of ADP-ribose. A declustering potential of 66 Volts was shown to give the highest signal to noise ratio for ADP-ribose. The entrance and collision cell exit potentials were both set to 10 Volts. Given these observations the parent ion mass of acetyl-ADPr was calculated to be 602.2 amu since acetyl-ADPr and ADP-ribose differ by an acetyl group which increases the mass of ADP-ribose by 42 amu.

Next, ADP-ribose was dissolved at a concentration of 1.0 micromolar in an assay buffer consisting of 50 mM Tris pH 7.5 containing 137 mM NaCl, 2.7 mM KCl, 1 mM MgCl₂, 0.1% BSA, 1 mM NAD⁺. Separation conditions to purify ADP-ribose from the assay buffer for mass spectrometric analysis were developed on a RapidFire® 200 High-Throughput Mass Spetrometry system. The optimized conditions for sample desalting were determined to be a sample wash with water for 3 seconds at a flow rate of 1.5 mL/min and sample elution with a 2:1:1 solution of water:acetonitrile:acetone containing 5 mM ammonium acetate for 3.5 seconds at a flow rate of 1.25 mL/min. A 4.0 μL bed volume RapidFire® separation cartridge containing a graphitic carbon phase (Hypercarb, ThermoFisher Scientific) was used for the sample purification. A 10 μL injection was used in all experiments. Source conditions used in the AB/Sciex API4000 mass spectrometer were as follows: Temperature=650 C; ionization voltage=5500 V; GS1 and GS2=50; Curtain Gas=20, dwell time=100 msec. Resolution of Q1 was set to Unit and Q3 set to low.

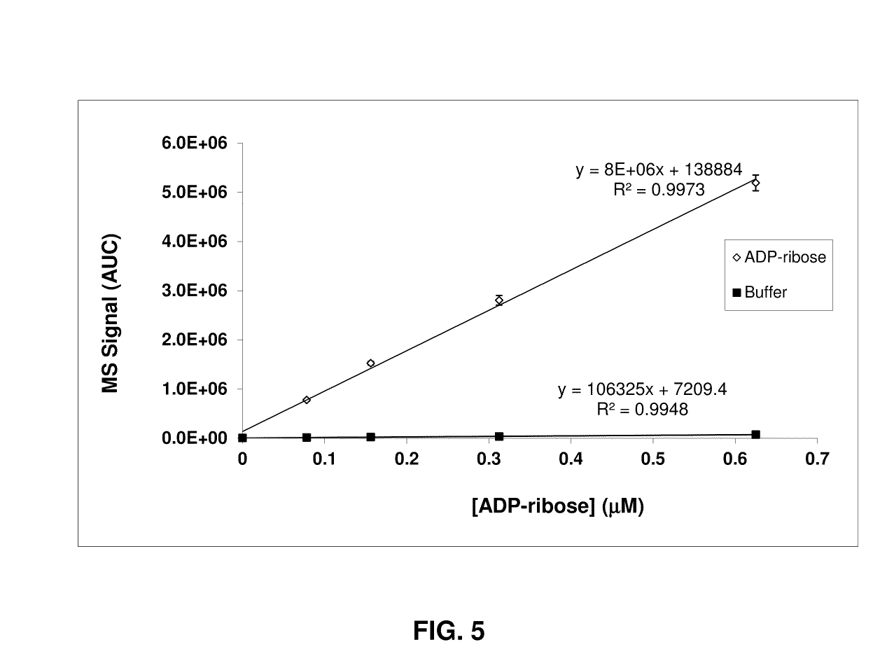

Using these optimized conditions a dilution series of ADP-ribose containing 625 nM, 312 nM, 156 nM, and 78 nM was prepared. The background signal was determined using injections of assay buffer without the addition of ADP-ribose. A total of 6 injections for each concentration were made and each injection of sample was followed by an injection of assay buffer to determine the carryover observed in the RapidFire® 200 system. The average signal intensity of the 6 sample and assay buffer injections are shown in FIG. 5. As can be seen under these conditions the signal was linear up to 625 nM and the limit of detection is below 1% formic acid and 1.0 μM ADP-ribose as an internal standard for the analytical measurement. The final ADP-ribose concentration was 0.5 μM as a result of the 2-fold dilution and is within the linear range for the analytical method. The reactions were stopped with addition of quench solution at 15, 30, 45, and 60 minutes as well as a zero minute control in which quench solution was added prior to initiation with substrate. A total of four different dilutions of the commercially available enzyme prep were used to determine the effect of enzyme concentration on the production of acetyl-ADPr.

### Example 2

### Characterization of acetyl-ADPr using RapidFire and Mass Spectrometry

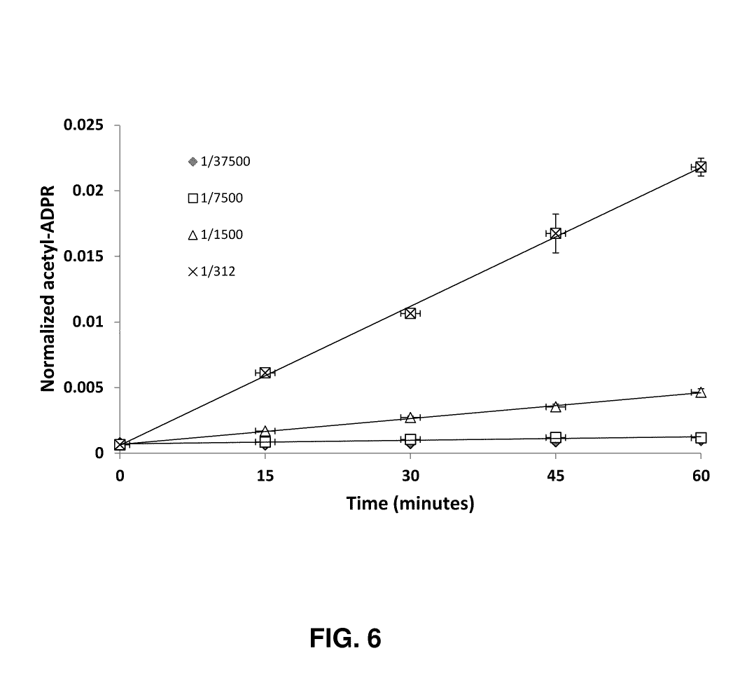

The RapidFire and mass spectrometry methods described in the current invention were used in the quantification of acetyl-ADPr product and ADP-ribose internal standard. The results of the time course experiment are shown in FIG. 6 and display a linear relationship between product formation and time at the 4 enzyme dilutions tested.

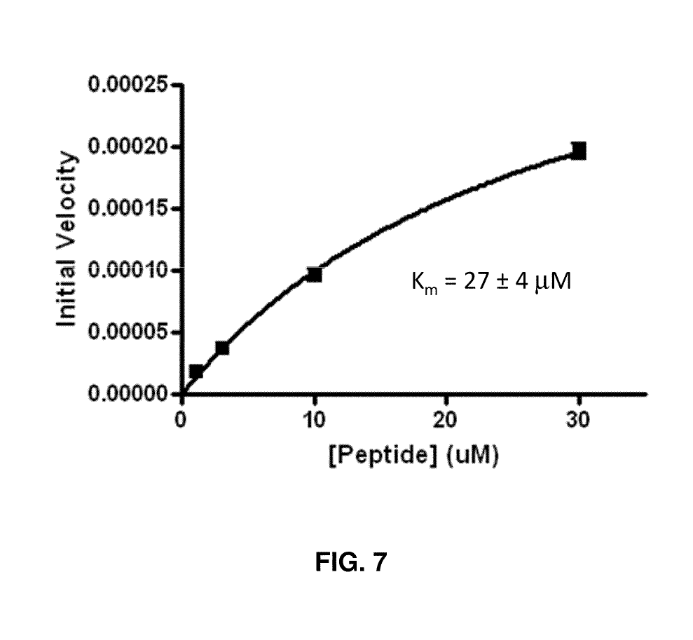

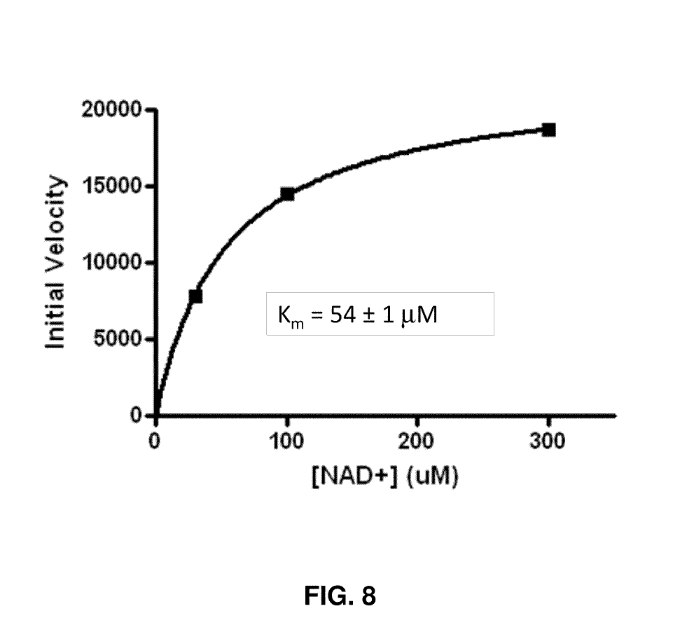

The Kₘ values for the two substrate of the reaction, the peptide substrate and NAD⁺, a series of experiments were performed where a range of concentrations of each substrate were varied. The result of the Km experiments for the peptide substrate and NAD⁺ are shown in FIG. 7 and in FIG. 8, respectively. The determine the Km value for the peptide substrate, reactions were run at room temperature in 50 mM Tris pH 7.5 containing 137 mM NaCl, 2.7 mM KCl, 1 mM MgCl₂, 0.1% BSA, 1 mM NAD⁺, and a 1/1500 dilution of enzyme. Reactions were stopped at various time points up to 2.5 hours by the addition of 75 mL 1% formic acid containing 100 nM ADPR. Similarly, to determine the Kₘ value for NAD⁺ in this assay an experiment was performed where reactions were run at room temperature in 50 mM Tris pH 7.5 containing 137 mM NaCl, 2.7 mM KCl, 1 mM MgCl₂, 0.1% BSA, 10 mM substrate, and a 1/1500 dilution of enzyme. Reactions were stopped at various timepoints up to 2 hours by the addition of 75 mL 1% formic acid. In both experiments, the RapidFire and mass spectrometry methods described in the current invention were used in the quantification of acetyl-ADPr product and ADP-ribose internal standard. The Kₘ values for the peptide substrate and NAD+were determined to be 27±4 μM and 54±1 μM, respectively.

### Example 3

### Characterization of SIRT Modulators

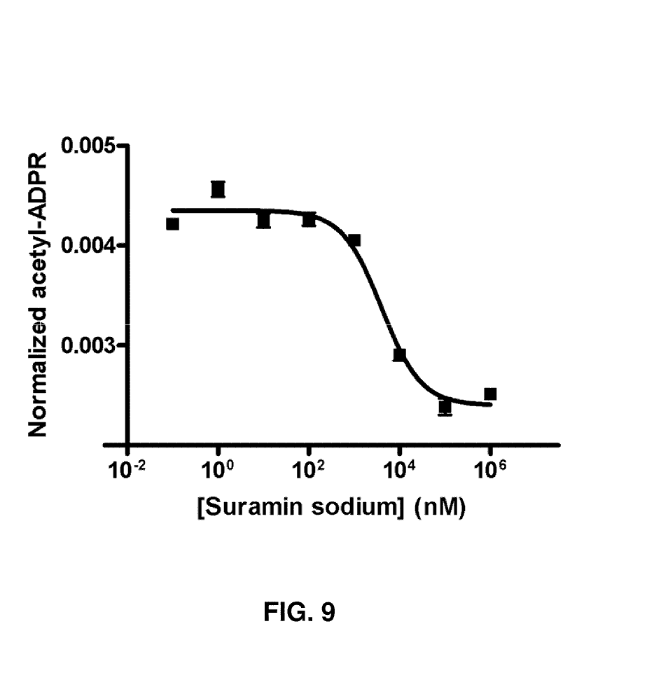

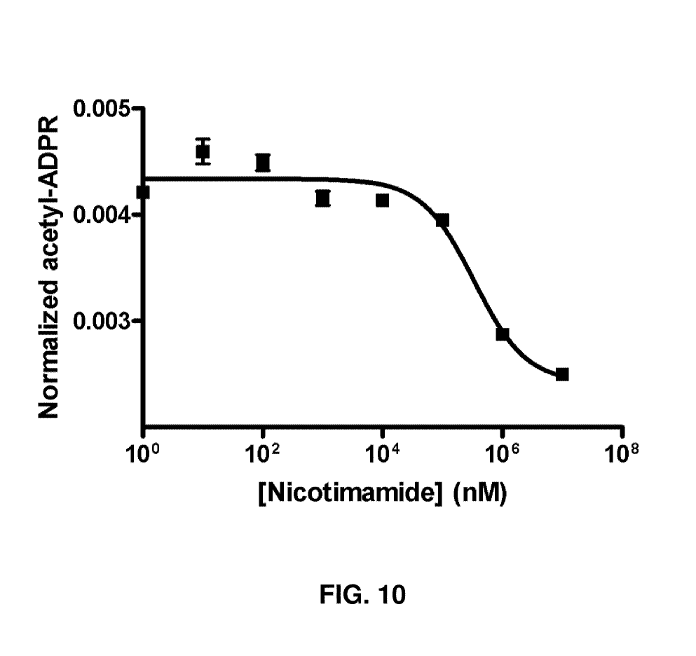

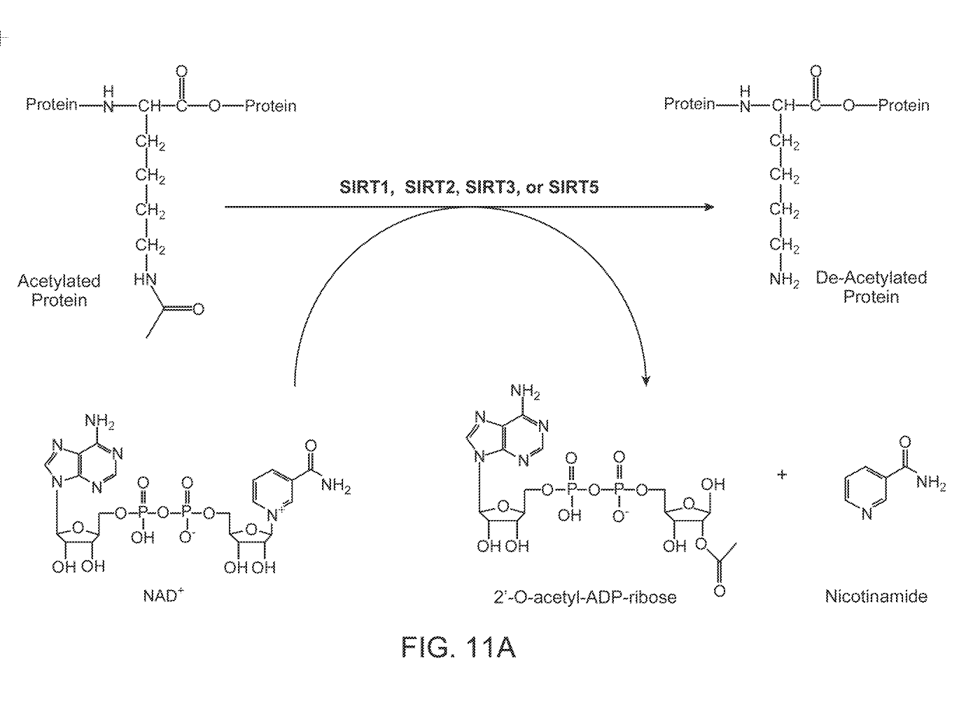

The invention provides methods for identifying agents that modulate the activity of NAD⁺-dependent Sirts as evidenced by the characterization of SIRT1 inhibitors. The effects of two known inhibitors of the SIRT1 enzyme were determined using the methods described in the current invention. The two test commercially available compounds chosen for this experiment were suramin sodium and nicotinamide. A log dilution series of each test compound starting at 1 mM for suramin sodium and 10 mM for nicotinamide, respectively, were prepared. For each of the two test compounds reactions were run at room temperature in 50 mM Tris pH 7.5 containing 137 mM NaCl, 2.7 mM KCl, 1 mM MgCl₂, 0.1% BSA, 10 mM substrate, and a 1/1500 dilution of enzyme. Reactions were stopped at 60 minutes by the addition of 75 mL 1% formic acid containing 100 nM ADPR. The RapidFire and mass spectrometry methods described in the current invention were used in the quantification of acetyl-ADPr product and ADP-ribose internal standard. The results of the experiments for are shown in FIG. 9 and in FIG. 10. The IC₅₀ values for suramin sodium and nicotinamide were determined to be 4.0±0.1 μM and 335 μM, respectively.

### Example 4

### Characterization of NAD⁺ Dependent SIRT1, SIRT2, AND SIRT3

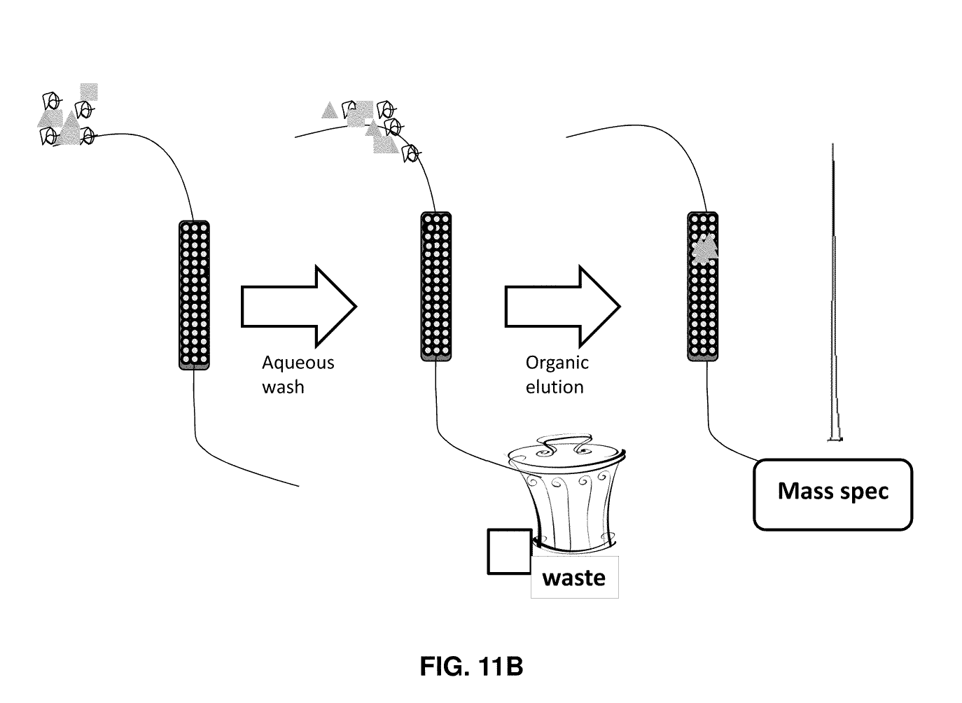

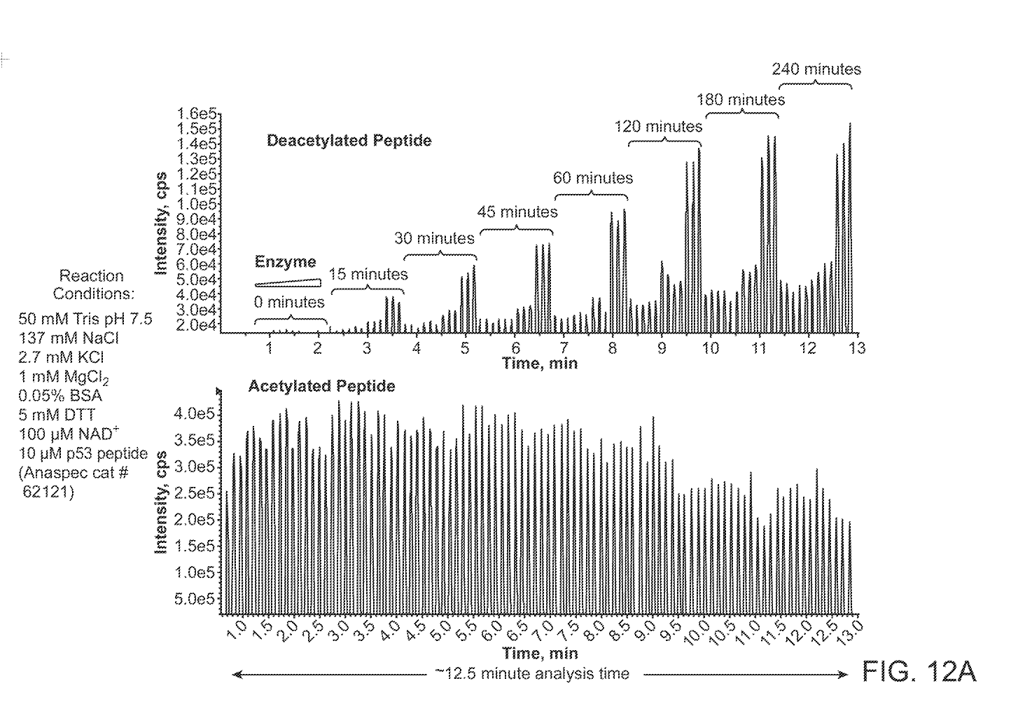

All NAD⁺ dependent protein deacetylases produce acetyl-ADPr as a coproduct of the deacetylation reaction. Thus, the present methods provide for the detection of modulators of all NAD⁺ dependent protein deacetylases, including SIRT1, SIRT2, SIRT3 and SIRT5 (FIG. 11A). Enzymes and substrates are commercially available. Samples can be assayed using the RapidFire RF-200 system connected to an AB/Sciex API-4000 triple quadrupole mass spectrometer. FIG. 11B is a schematic diagram illustrating the SPE-MS/MS Analysis.

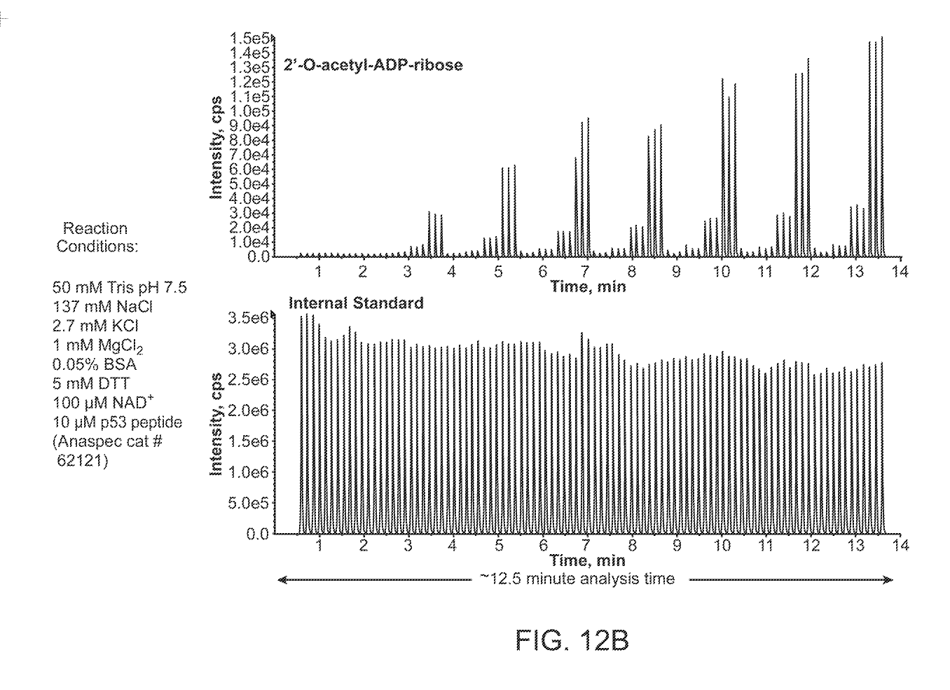

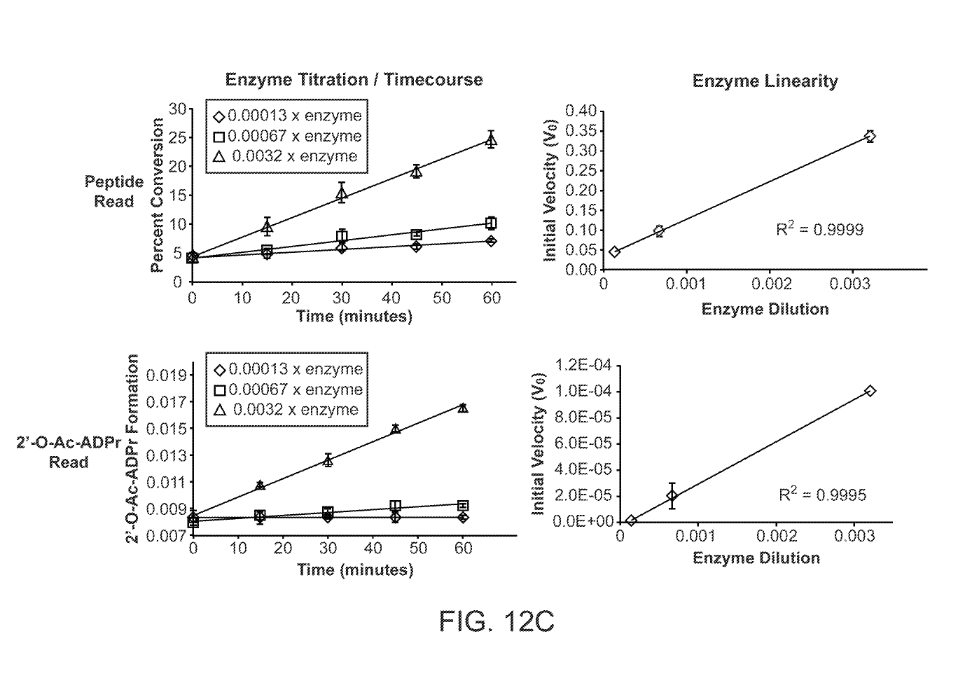

The methods provided by the present invention provide high-throughput methods that can be carried out much more efficiently than methods that relied on peptide based analysis (FIGS. 12A and 12B) without any reduction in the quality of the analysis (FIG. 12C)

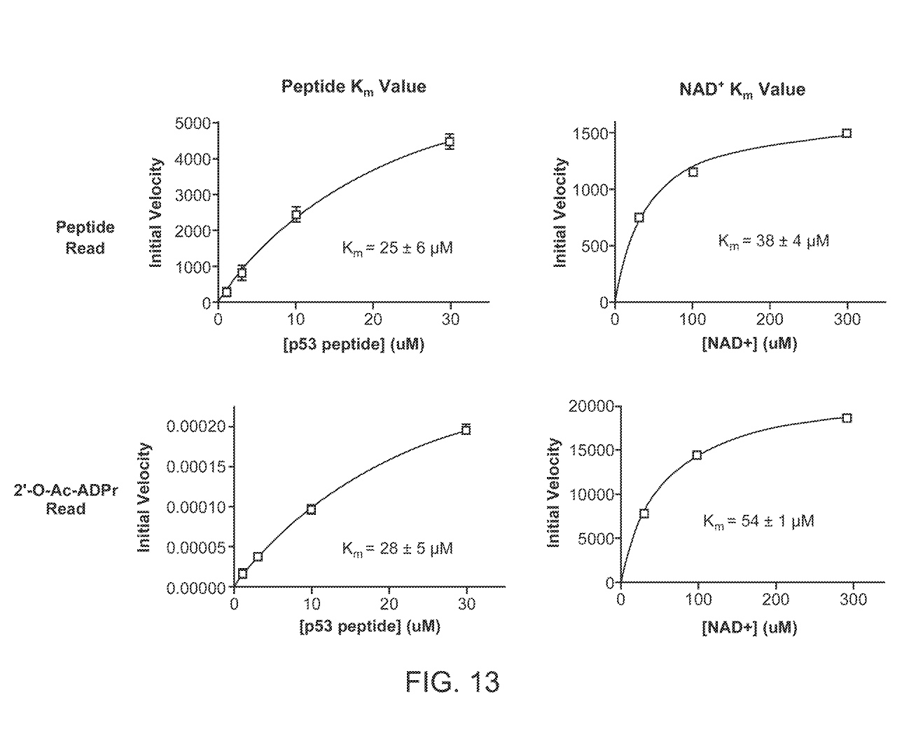

FIG. 13A provides a comparison of the K_{M} determinations for p53 peptide and NAD co-substrates using peptide-based and acetyl-ADPr product based detection methods.

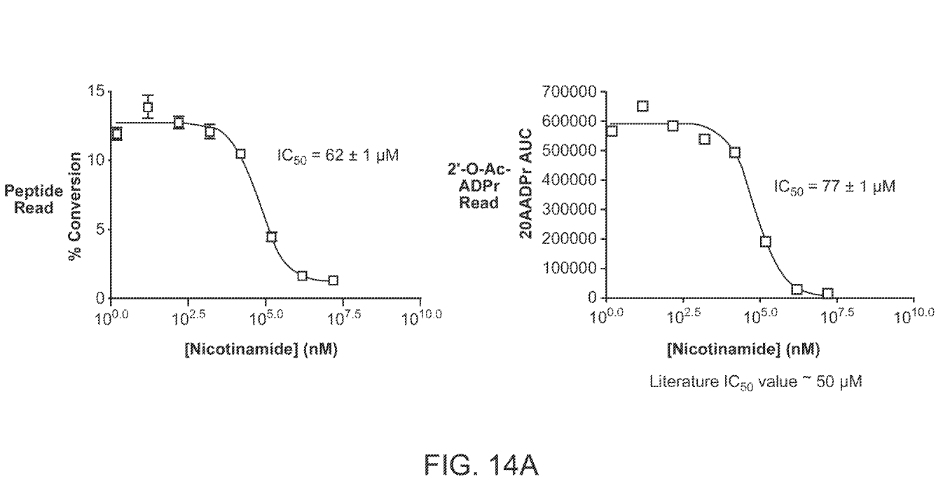

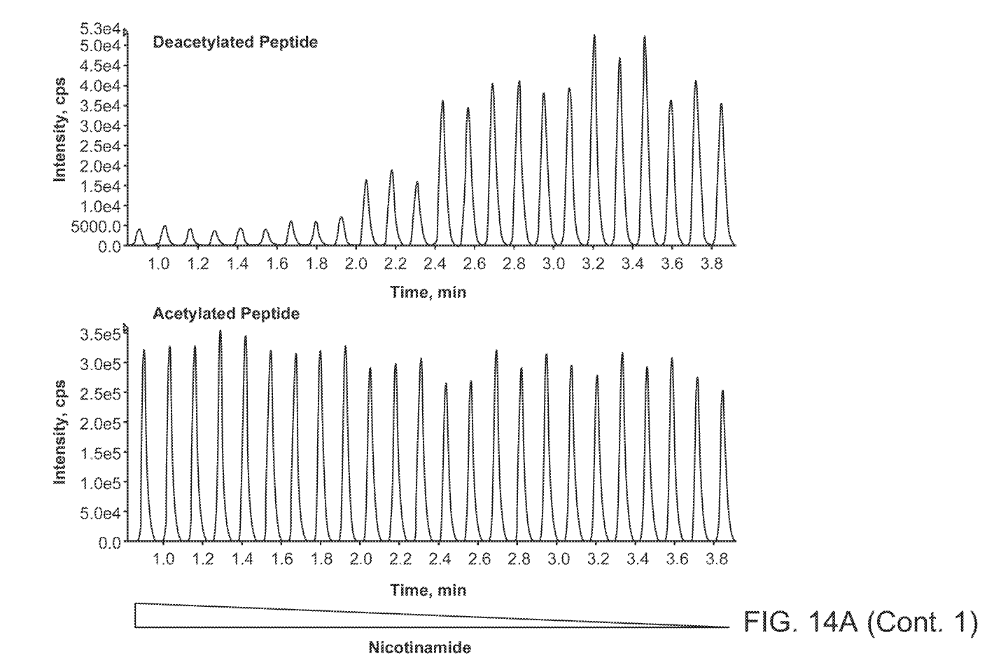

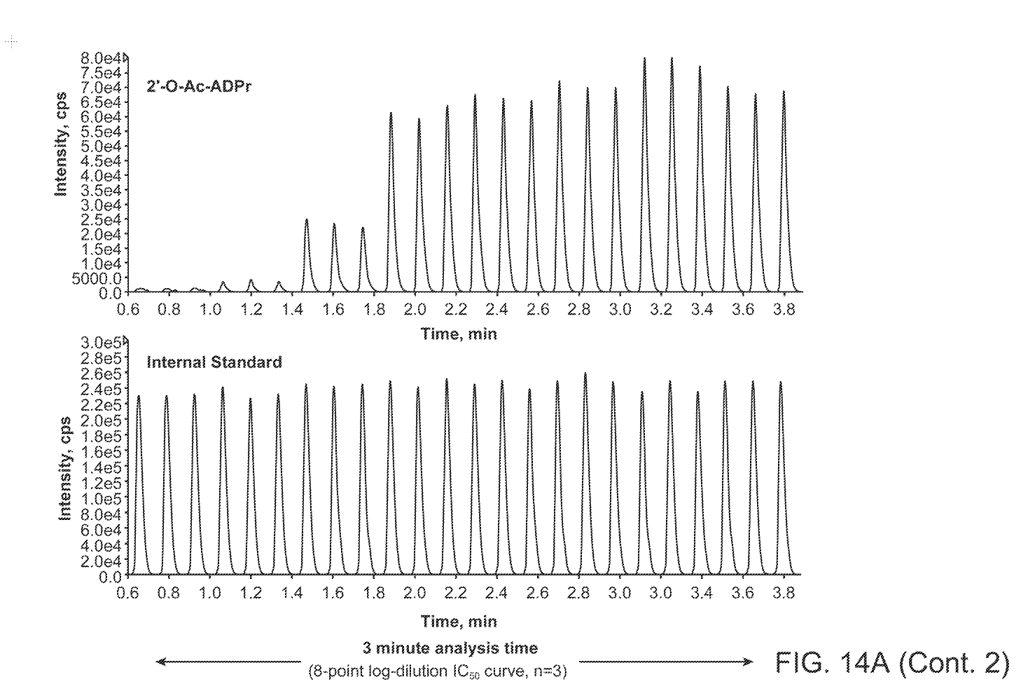

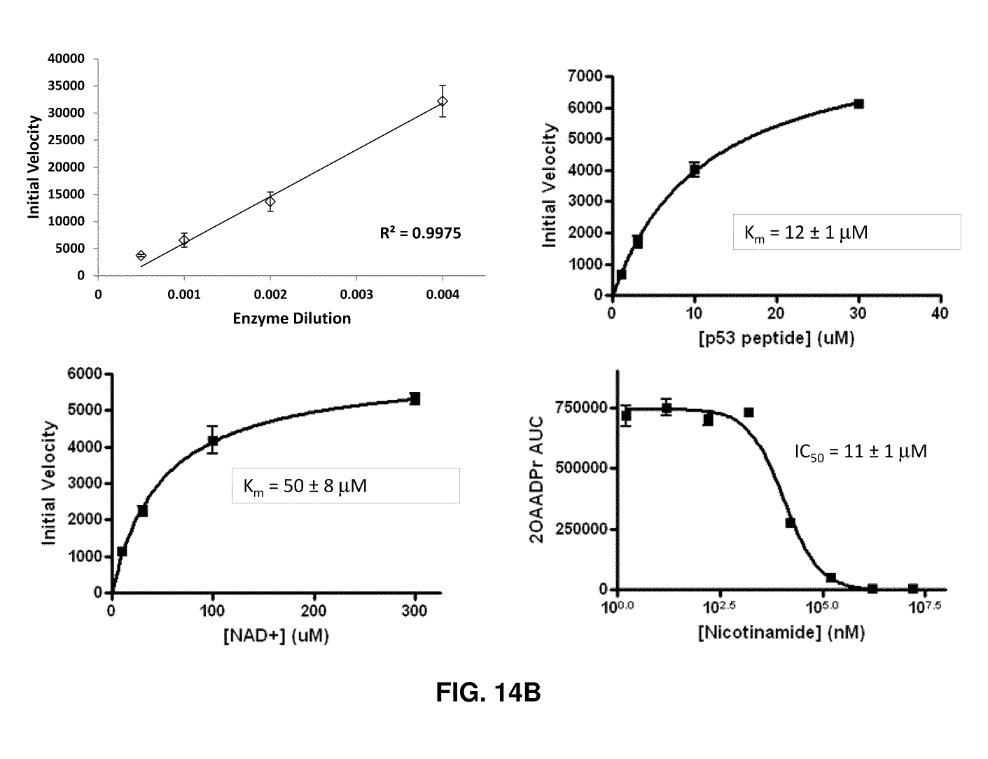

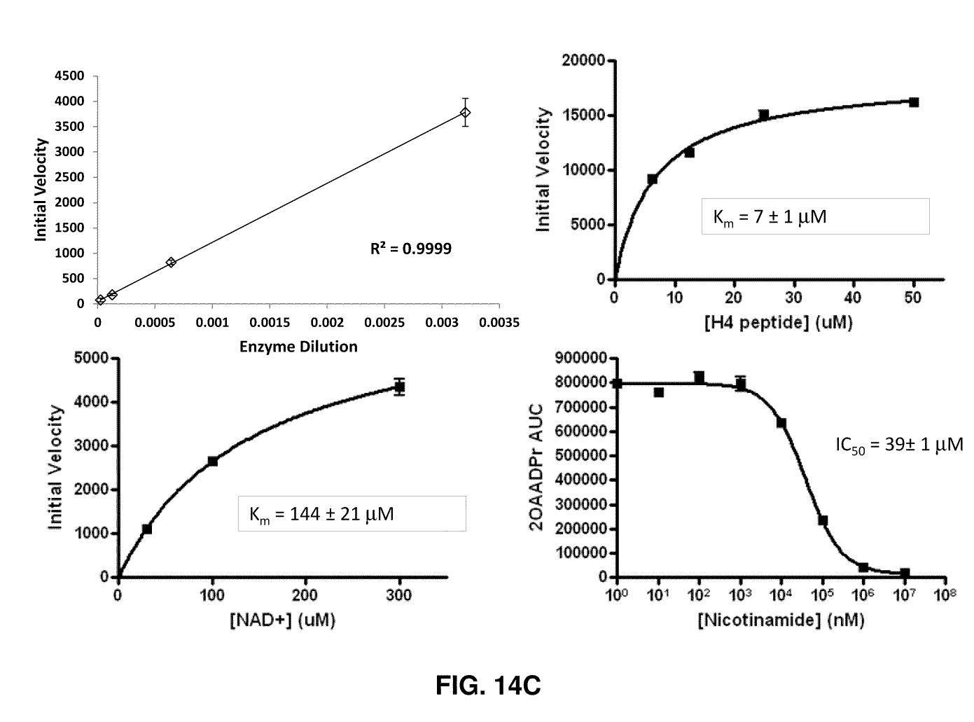

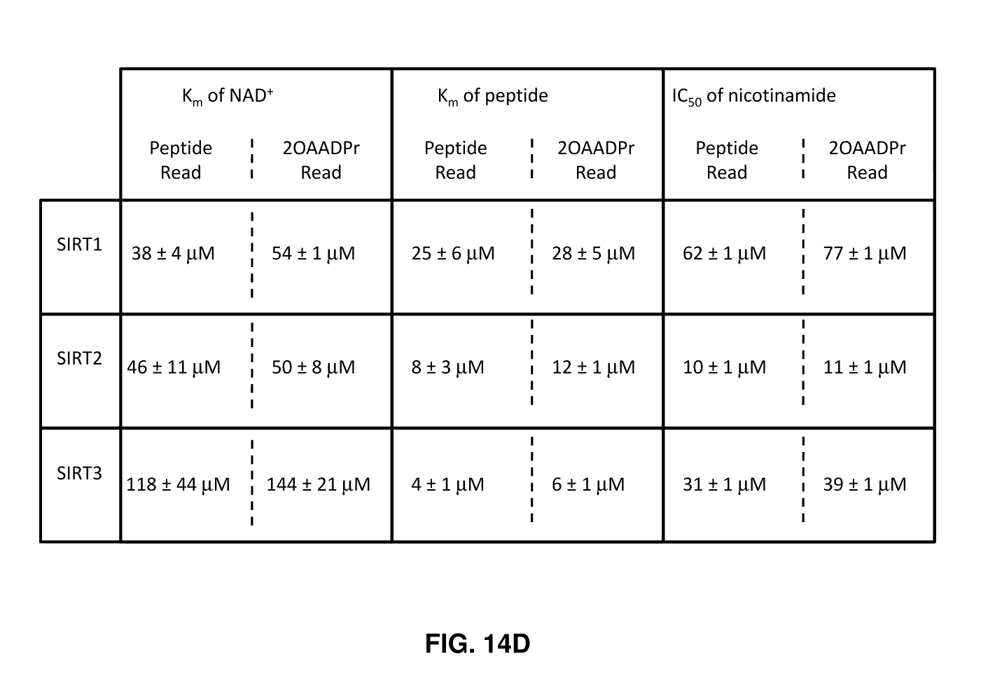

FIGS. 14A, B, C and D provide a comparison of peptide-based and acetyl-ADPr product based detection methods for SIRT1, SIRT2, and SIRT3.

### Example 5

### Other Embodiments

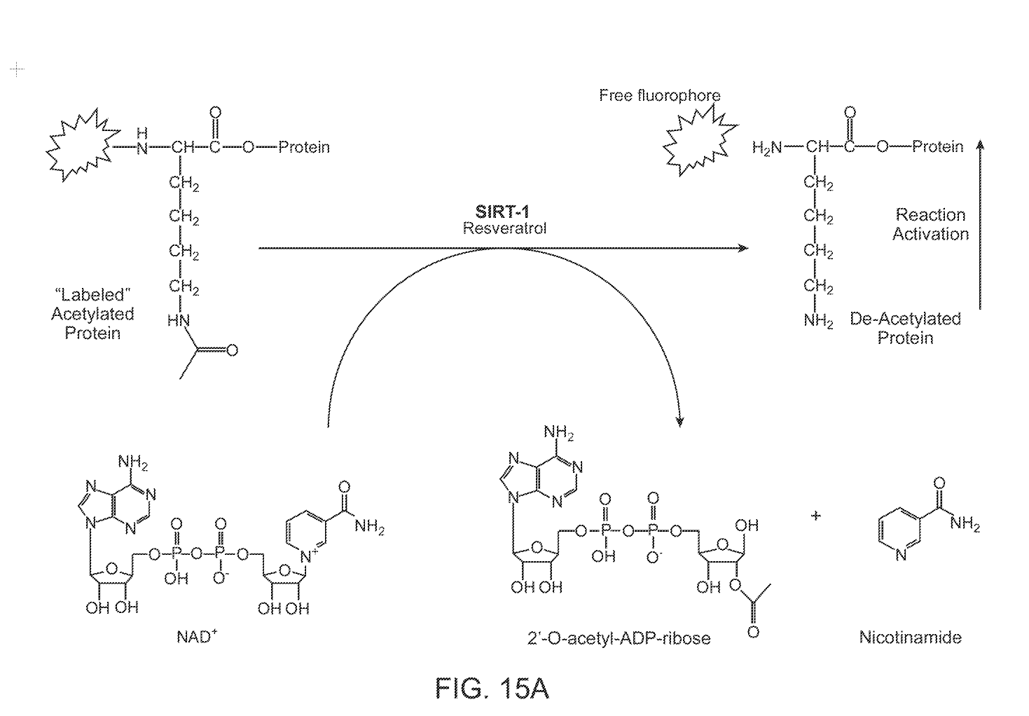

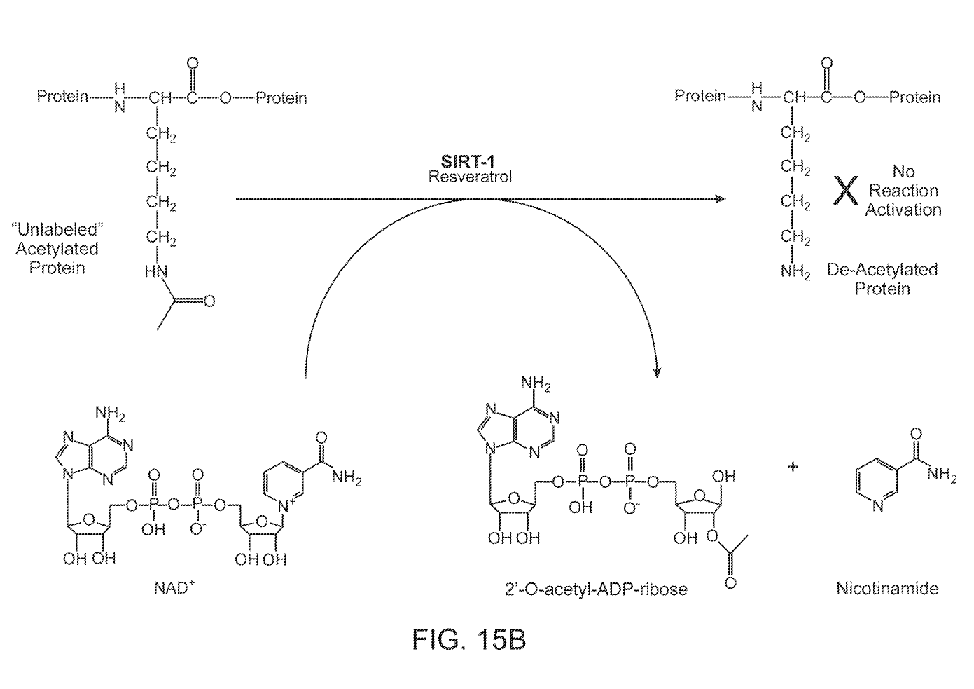

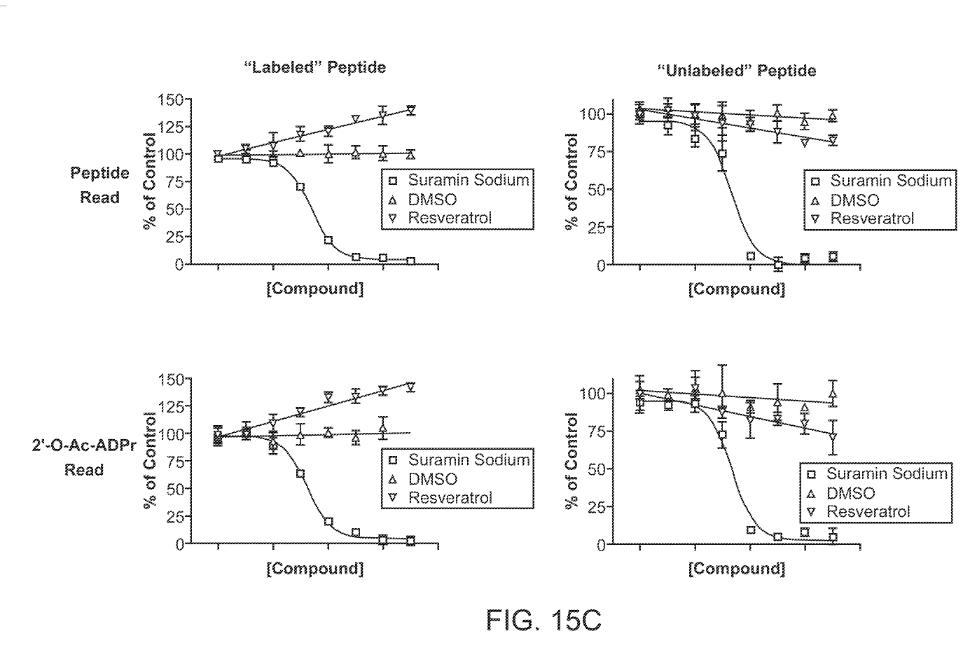

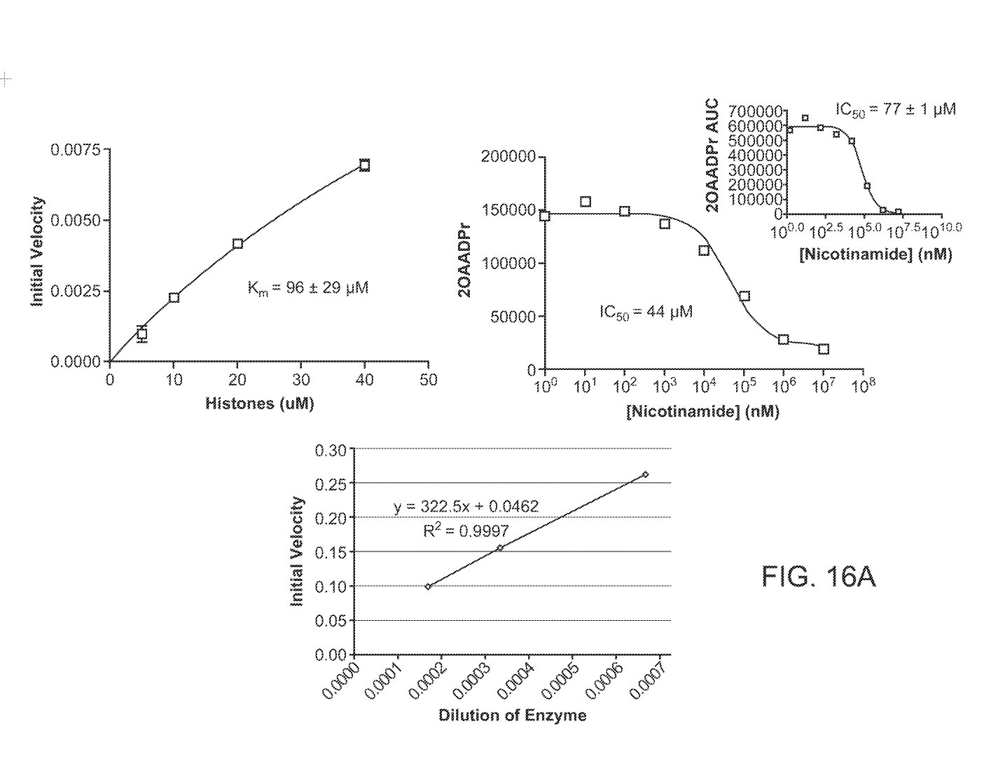

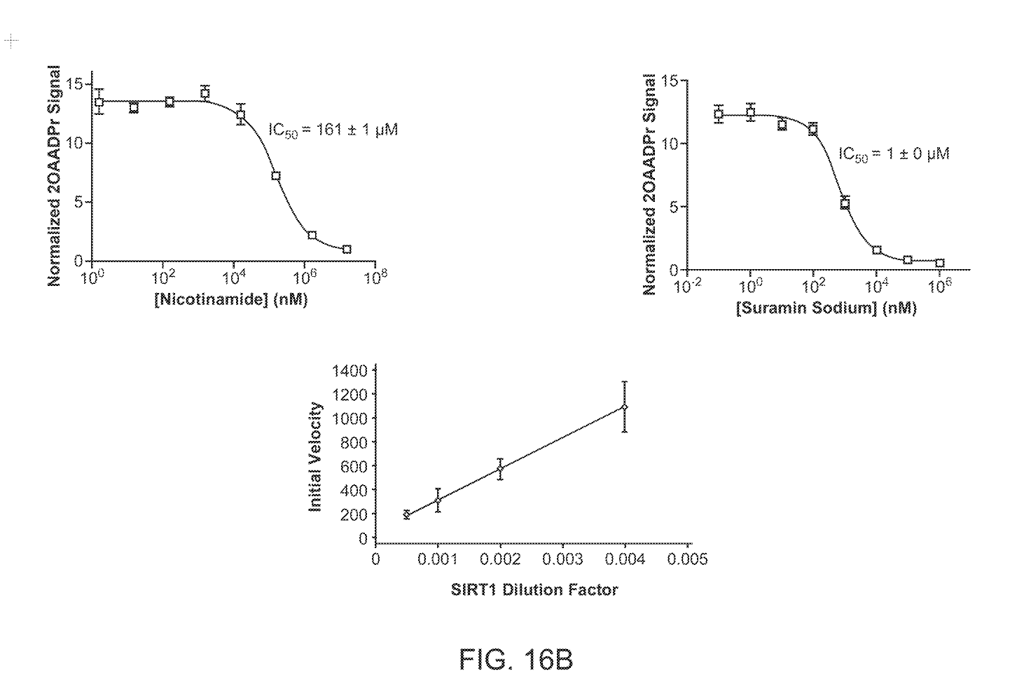

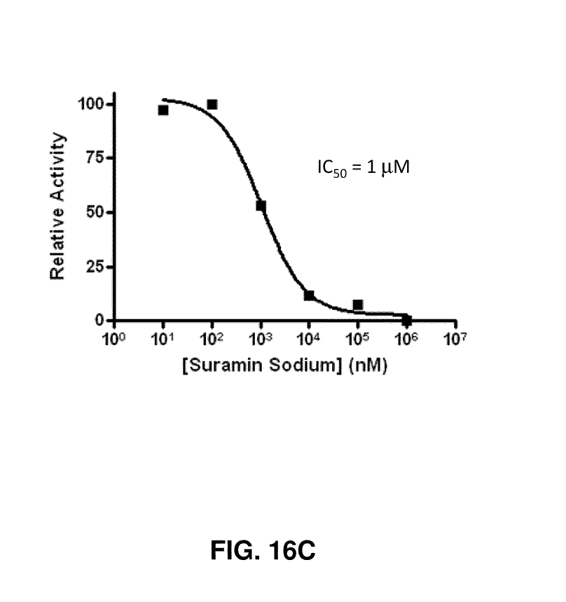

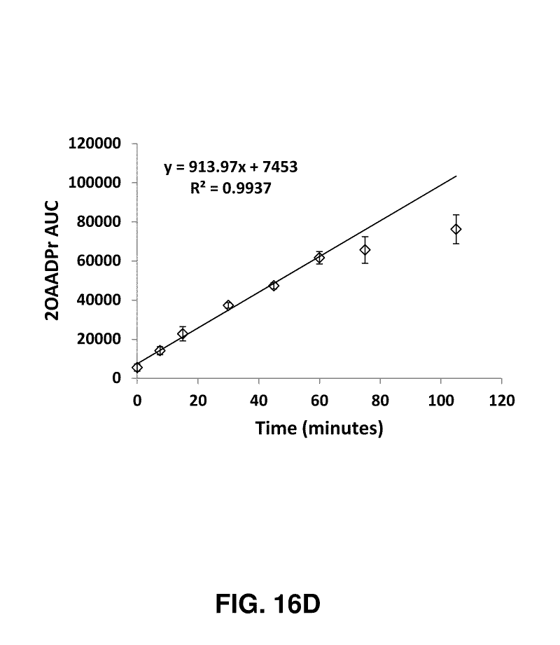

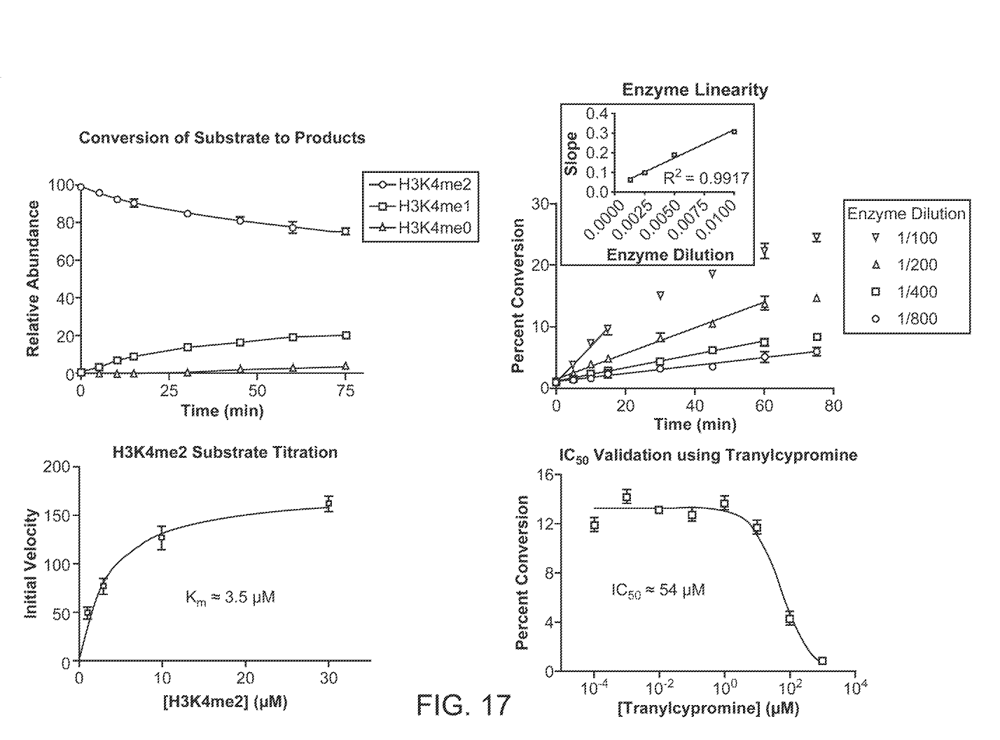

The methods described herein can be used to detect sirtuin activation as shown in FIGS. 15A-15C. The methods can also provide fast, convenient and accurate way for detecting the deacetylation of whole protein substrates as shown in FIGS. 16A-16C. The methods may also be used for epigenetic screen as shown in FIG. 17, which describes an LSD-1 Histone Demethylase assay.

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for detecting NAD⁺ dependent protein deacetylase activity, the method comprising contacting a substrate with a NAD⁺ dependent protein deacetylase and detecting O-acetyl-ADP-ribose using mass spectrometry.

2. The method of claim 1, wherein said contacting is done in the presence of a test agent and wherein an alteration in the level of O-acetyl-ADP-ribose as compared to a control indicates that said test agent modulates said NAD⁺ dependent protein deacetylase.

3. The method of claim 1, wherein the NAD⁺ dependent protein deacetylase is SIRT1, SIRT2, SIRT3 or SIRT5.

4. The method of claim 1, wherein the substrate is a protein, peptide, or protein complex.

5. The method of claim 1, wherein said mass spectrometry is done by a high-throughput mass spectrometry system.

6. The method of claim 1, wherein the level of O-acetyl-ADP-ribose detected is indicative of the level of deacetylated substrate.

7. The method of claim 5, wherein the O-acetyl-ADP-ribose detected is at a molar ratio of 1:1 with a deacetylated substrate.

8. The method of claim 1, wherein the method is carried out in the presence of an internal standard.

9. The method of claim 1, wherein the internal standard is ADP ribose.

10. The method of claim 1, wherein the substrate is a histone, an HMG protein, p53, c-Myb, GATA-1, EKLF, MyoD, E2F, dTCF, or HIV Tat, or a fragment thereof.

11. The method of claim 2, wherein the test agent activates or inhibits said NAD⁺ dependent protein deacetylase.

12. The method of claim 11, wherein the test agent activates a sirtuin.

13. The method of claim 12, wherein the test agent activates the sirtuin to a greater extent than resveratrol.

14. The method of claim 13, wherein a compound that has sirtuin activating activity at least 5-fold greater than the sirtuin activating activity of resveratrol is identified.

15. The method of claim 1, wherein the mass spectrometry is electrospray ionization (ESI) mass spectrometry or matrix-assisted laser desorption/ionization (MALDI) mass spectrometry.

16. The method of claim 1, wherein the test agent is a small molecule, polypeptide or polynucleotide.

17. A kit comprising:
a NAD+ dependent protein deacetylase enzyme;
a substrate for said NAD⁺ dependent protein deacetylase; and
instructions for using said NAD+ dependent protein deacetylase enzyme and said substrate for said NAD⁺ dependent protein deacetylase in the method of claim 1.

18. The kit of claim 17, wherein the NAD⁺ dependent protein deacetylase is SIRT1, SIRT2, SIRT3 or SIRT5.

19. The kit of claim 17, wherein the substrate is a histone, an HMG protein, p53, c-Myb, GATA-1, EKLF, MyoD, E2F, dTCF, or HIV Tat, or a fragment thereof.

20. The kit of claim 17, further comprising a positive control.
